(19) United States
(12) Patent Application Publication
(10) Pub. No.: US 2010/0120703 A1
(43) Pub. Date: May 13, 2010

(54) COMPOSITIONS AND METHODS FOR TREATING CANCER

(75) Inventors: John Wing Shing Ho, Hong Kong (CN); Jill Jie Meng, Hong Gong (CN)

Correspondence Address: KNOBBE MARTENS OLSON & BEAR LLP, 2040 MAIN STREET, FOURTEENTH FLOOR, IRVINE, CA, 92614, US

(73) Assignee: THE CHINESE UNIVERSITY OF HONG KONG, Hong Kong (JP)

(21) Appl. No.: 12/529,165

(22) PCT Filed: Mar. 7, 2008
(86) PCT No.: PCT/CN2008/000460

§ 371 Date: Aug. 28, 2009

Related U.S. Application Data
(60) Provisional application No. 60/894,132, filed on Mar. 9, 2007.

(51) Int. Cl. A61K 31/70; A61P 35/00; A61P 1/16

(52) U.S. Cl. 514/24

(57) ABSTRACT

Pharmaceutical compositions comprising sinigrin and a pharmaceutically acceptable carrier and use thereof for treating liver cancer. A method for treatment of liver cancer in a subject comprising administering to the subject in need thereof and suffering from cancer, a pharmaceutically effective amount of sinigrin, is also provided.

### FIELD OF THE INVENTION

The present invention relates to a pharmaceutical composition comprising sinigrin or a pharmaceutically acceptable derivate thereof, or a mixture of both, and use thereof in treating cancer.

### BACKGROUND OF THE INVENTION

Human liver cancer (hepatoma) is among the top 12% in cancer deaths and cannot be easily cured at its later stages. Because conventional chemotherapeutic agents used to treat hepatoma (e.g., taxol, cisplatin, doxorubicin) in early stages of development have considerable side effects, their therapeutic benefits are limited. In addition, these agents are expensive. Both environmental and genetic factors play an important role in hepatocellular carcinoma development in the liver. In rat experimental models, it has been demonstrated that dietary carcinogens increased susceptibility to liver cancer. Considerable research has focused on relieving symptoms of liver disorders, but not treatment, due to a lack of hepatoma-specific drugs and the side effects that result from long-term administration of conventional chemotherapeutic agents. These liver conditions can produce tremendous discomfort and pain in patients. Ongoing research is directed to finding a curative agent for the treatment and suppression of liver cancer from metastasis using a pure active component.

Traditional Chinese medicines (TCMs) have been reported to have multiple pharmacological actions. TCMs have anti-inflammatory activity against human and rat tumor cells in the liver, and some TCMs show inhibitory effects on the proliferation of cancer cells (Alshatwi A A, Han C T, Schoene N W &Lei K Y. (2006) Nuclear Accumulations of p53 and Mdm2 Are Accompanied by Reductions in c-Abl and p300 in Zinc-Depleted Human Hepatoblastoma Cells. Exp Biol Med (Maywood). 231(5):611-618). Early studies have shown that TCMs could potentiate the anti-tumor activity of cyclophosphamide and radiation in animals. However, none of these herbs have been used alone for treatment of liver cancer.

Sinigrin is a glucosinolate which belongs to the family of glucosides found in some plants of the Brassica family such as brussel sprouts, broccoli and the seeds of black mustard (Brassica nigra). Sinigrin is a unique pure compound with a low molecular weight of 397.46, and a chemical formula of C₁₀H₁₆NO₉S₂.K.

U.S. patent application Ser. No. 09/952,478, the entire contents of which are incorporated herein by reference, discloses a pharmaceutical composition for inhibiting cancer cell proliferation comprising canola extracts selected from the group consisting of a phenolic acid, a carotenoid, a tocopherol/sterol, and a glucosinolate, in which the phenolic acid is the most active ingredient in the composition. Although sinigrin is disclosed as one of 12 species of glucosinolate, it fails to disclose the effect of sinigrin as an active ingredient in the canola extracts.

Johnson I., et al. (Colon cancer proliferation desulfosinigrin in Wasabi (Wassabia japonica). Nutrition and Cancer. 2004; 48(2):207) explored the effect of sinigrin on the intestinal mucosa of rats previously treated with dimethylhydrazine (DMH) and found that sinigrin could induce a higher level of apoptosis in colonic tissue from DMH treated rats compared with those given DMH only, and sinigrin administered after DMH suppresses induction of aberrant crypt foci in colonic tissue. However, Zheng, Q, et al. (Further investigation of the modifying effect of various chemopreventive agents on apoptosis and cell proliferation in human colon cancer cells. Journal of Cancer Research and Clinical Oncology, 2002; 128:539-546) reported that sinigrin might be apoptosis- and cell proliferation-independent. Thus, there is conflicting information on the action of sinigrin in colon cancer cells. In addition, the effect of sinigrin on cancer cells derived from different tissues has not been described.

The present invention provides a pharmaceutical composition comprising sinigrin and uses thereof for treating cancer.

### SUMMARY OF THE INVENTION

One aspect of the invention provides a pharmaceutical composition for treating cancer consisting sinigrin or a pharmaceutically acceptable derivate thereof or a mixture of both, and a pharmaceutically acceptable carrier.

In embodiments of the invention, the composition can comprise a therapeutically effective amount of sinigrin or a pharmaceutically acceptable derivate thereof, or a mixture of both.

Another aspect of the invention provides a method for treating cancer with sinigrin or a pharmaceutically acceptable derivate thereof or a mixture of both, or a pharmaceutical composition provided herein. The method, for example, can comprise administering a therapeutically effective amount of sinigrin or a pharmaceutically acceptable derivate thereof or a mixture of both, or a pharmaceutical composition described herein to a subject at risk of developing or suffering from cancer.

Another aspect of the invention provides use of sinigrin or a pharmaceutically acceptable derivate thereof or a mixture of both in manufacturing a medicament or a pharmaceutical composition for treatment of cancer.

Another aspect of the invention provides a method of inhibiting growth of hepatoma cells comprising contact the hepatoma cells with a pharmaceutical composition described herein or sinigrin or a pharmaceutically acceptable derivate thereof or a mixture of both.

Another aspect of the invention provides a method of inducing apoptosis of hepatoma cells comprising contact the hepatoma cells with a pharmaceutical composition described herein or sinigrin or a pharmaceutically acceptable derivate thereof or a mixture of both.

In one embodiment, the pharmaceutically acceptable carrier is water.

In one embodiment, sinigrin or a pharmaceutically acceptable derivate thereof or a mixture of both is the only active component in the pharmaceutical composition of the invention.

In another embodiment of the invention, sinigrin or a pharmaceutically acceptable derivate thereof or a mixture of both can be administered in combination with other agents, or the pharmaceutical composition may comprise other agents.

In some embodiments, the subject to be treated is a mammal. In preferred embodiments, the subject is a human.

In an embodiment of the invention, the cancers to be treated include liver cancer, pancreatic cancer and lung cancer. In preferred embodiments, the cancer is liver cancer.

The cancers to be treated may be primary or secondary, at promotion stage or at progression stage. In some embodiments where the cancers are at progression stage, the drug not only eliminates or minimizes the primary tumors, but also suppress metastasis of tumor cells, i.e. the secondary cancer.

In embodiments of the invention, the composition or medicament can be administered via any suitable routes as needed, such as oral administration, injection and infusion. In preferred embodiments, the composition or medicament is administered orally.

In an embodiment of the invention, the therapeutically effective amount of sinigrin or a pharmaceutically acceptable derivate thereof or a mixture of both is in the range of from about 0.1 mg/kg to about 300 mg/kg body weight daily. In other embodiments, the therapeutically effective amount of sinigrin is in the range of from about 1 mg/kg to 100 mg/kg body weight daily. In other embodiments, the therapeutically effective amount of sinigrin is about 10 mg/kg body weight daily.

Without wishing to be bound by any particular theory, sinigrin or a pharmaceutical composition comprising sinigrin can treat cancer by inducing G0/G1 phase arrest in cell cycle and/or apoptosis of tumor cells, such as human hepatoblastoma cells or liver cancer cells.

### BRIEF DESCRIPTION OF THE DRAWINGS

The accompanying drawings, which are incorporated in and form part of the specification, merely illustrate certain preferred embodiments of the present invention. Together with the remainder of the specification, they are meant to serve to explain preferred embodiments of the invention to those of skilled in the art.

FIG. 1 is a graph showing the viability of HepG2, WRL-68 and Clone 9 cells after SIN treatment for 72 hours. The dark line shows the viability of the HepG2 cells; the dark grey line shows the viability of the Clone 9 cells; and the light grey line shows the viability of the WRL 68 cells.

FIGS. 2A-2L are DNA histograms of fluorescence activated cell sorting (FACS) demonstrating the distribution of HepG2 cells in different phases of the cell cycle after SIN treatment for different length of time. Panels: A—SIN (sinigrin) 0 mM/24 hours, B—SIN 0.1 mM/24 hours, C—SIN 0.5 mM/24 hours, D—SIN 0 mM/48 hours, E—SIN 0.1 mM/48 hours, F 0.5 mM/48 hours, G—SIN 0 mM/72 hours, H—SIN 0.1 mM/72 hours, I—SIN 0.5 mM/72 hours, J—SIN 0 mM/96 hours, K—SIN 0.1 mM/96 hours, L—SIN 0.5 mM/96 hours.

FIGS. 3A-3D are graphs showing cell distribution in different phases of the cell cycle after SIN treatment at different time points. Panels: A—24 hour incubation of SIN, B—48 hour incubation of SIN, C—72 hour incubation of SIN, D—96 hour incubation of SIN.

FIG. 4 shows DNA fragmentation of HepG2 cells treated with different concentrations of SIN for 96 hours. Lane A—1 kb DNA marker, Lane B—0.25 mM SIN treatment, Lane C—0.5 mM SIN treatment, Lane D—0 mM SIN, control.

FIG. 5A is a scatter plot of gene fold difference showing the effects of SIN on gene expression. The x-axis represents control gene transcription, and the y-axis represents SIN treated cell gene transcription. The dark grey crosses represent those genes transcribed 3 fold up-regulated in the SIN treated cells. The light grey crosses represent those genes transcribed 3 fold down-regulated in the SIN treated cells.

FIGS. 5B-5C are membrane images of the cDNA array. Panels: A—Control membrane, B—SIN treated sample membrane.

FIG. 6 includes a bar chart showing the genes that are significantly up-regulated (light grey bars) or down-regulated (dark grey bars) in SIN treated HepG2 cells as well as an accompanying table listing the genes and their fold change in detail.

FIG. 7 is a graph outlining the experimental protocol for rat treatment in the promotion stage.

FIGS. 8A-8C are photos of rat livers at the promotion stage of HCC development, showing the results of the direct observation. Panels: A—Rat liver from the negative control group; B—Rat liver from the positive control group; C—Rat liver from the SIN-treatment group.

FIGS. 9A-9B are bar charts showing the effects of SIN on the weights of rat livers at the promotion stage (n=4). The results are expressed as means±SD, and the data obtained were evaluated by ANOVA. Statistical analyses of the data were performed using the student's t-test. The symbol “*” indicates that p<0.05. Panels: A—Liver weight/body weight index of the three groups, B—Liver weight/body weight index of the positive control and SIN-treatment groups compared with the negative control group. The negative control group was considered as 100%.

FIG. 10 is a bar chart showing the effects of SIN on the serum ALT and AST levels in rats at the promotion stage (n=4). The amount of ALT/AST in rat serum of positive control and SIN-treatment groups was compared with the negative control group. The negative control group was considered as 100%. The results are expressed as means±SD, and the data obtained were evaluated by ANOVA. Statistical analyses of the data were performed using the student's t-test. The symbol “*” indicates that p<0.05.

FIG. 11 is a graph summarizing the ABC staining protocol of immunostaining.

FIGS. 12A-12D are micrographs of rat liver sections showing the damaged hepatocyte structure in the liver from the positive control and the restored hepatocyte structure in the liver from the SIN—treatment group at the promotion stage. Panels: A—Rat liver section from the negative control group. Clear histological structure of the hepatocyte could be observed. B—Rat liver section from the positive control group. The hepatocyte lost the central vein. C—Rat liver section from the positive control group. Cytoplasmic vacuolization within the hepatocytes could be observed. D—Rat liver section from SIN-treatment group. The basic structure was restored.

FIGS. 13A-13G are micrographs of rat liver sections showing the GST-p positive areas at the promotion stage. The results are expressed as mean±SD, and the data obtained were evaluated by ANOVA. Statistical analyses of the data were performed using the student's t-test. Panels: A—Rat liver section from the negative control group (2.5×). No GST-p expression was found in the whole section. B—Rat liver section from the negative control group (20×). Normal basic structure could be observed. C—Rat liver section from the positive control group (2.5×). GST-p positive area could be found across the section. D—Rat liver section from the positive control group (10×). GST-p positive area appeared in clusters. E—Rat liver section from the SIN-treatment group (2.5×). Only limited GST-p positive area was found. F—Rat liver section from the SIN-treatment group (20×). Basic structure was restored. G—Rat liver section from the SIN-treatment group (20×).

FIG. 14 is a bar chart comparing the percentage of GST-p positive area/whole section area of each group at the promotion stage (n=4). The GST-p positive area/whole section area ratios of the positive control and SIN-treatment groups were compared to that of the negative control group. The negative control group was 0%. The symbol “*” indicates that p<0.05.

FIGS. 15A-15D show the mRNA expressions of p53 and Mdm2 in rats at the promotion stage (n=4). The results are expressed as mean±SD, and the data obtained were evaluated by ANOVA. Statistical analyses of the data were performed using the student's t-test. The symbol “*” indicates that p<0.05. Panels: A—p53 expression level measured by the ratio of p. 53 to β-actin. B—p53 mRNA expression measured by RT-PCR, C—Mdm2 expression level measured by the ratio of Mdm2 to β-actin D—Mdm2 mRNA expression measured by RT-PCR.

FIGS. 16A-16D show the expressions of total p53 protein and WT p53 protein in rats at the promotion stage (n=4). The results are expressed as mean±SD, and the data obtained were evaluated by ANOVA. Statistical analyses of the data were performed using the student's t-test. The symbol “*” indicates that p<0.05. Panels: A—Total p53 expression level measured by the ratio of total p53 to)₃-actin. B—Total p53 protein expression measured by Western Blot. C—WT p53 expression level measured by the ratio of WT p53 to β-actin. D—WT p53 protein expression measured by Western Blot.

FIGS. 17A-17B show the expression of Mdm2 protein in rats at promotion stage (n=4). The results are expressed as mean±SD, and the data obtained were evaluated by ANOVA. Statistical analyses of the data were performed using the student's t-test. The symbol “*” indicates that p<0.05. Panels: A—Mdm2 expression level measured by the ratio of Mdm2 to β-actin B—Mdm2 protein expression measured by Western Blot.

FIGS. 18A-18D show the expression of p21 protein and PCNA protein in rats at the promotion stage (n=4). The results are expressed as mean±SD, and the data obtained were evaluated by ANOVA. Statistical analyses of the data were performed using the student's t-test. The symbol “*” indicates that p<0.05. Panels: A—p21 expression level measured by the ratio of p21 to)₃-actin. B—p21 protein expression measured by Western Blot, C—PCNA expression level measured by the ratio of p21 to β-actin. D—PCNA protein expression measured by Western Blot.

FIGS. 19A-19D show the expression of Bax protein and Bcl-2 protein in rats at the promotion stage (n=4). The results are expressed as mean±SD, and the data obtained were evaluated by ANOVA. Statistical analyses of the data were performed using the student's t-test. The symbol “*” indicates that p<0.05. Panels: A—Bax expression level measured by the ratio of Bax to β-actin., B—Bax protein expression measured by Western Blot, C—Bcl-2 expression level measured by the ratio of Bcl-2 to β-actin., D—Bcl-2 protein expression measured by Western Blot.

FIG. 20 is a graph outlining the experimental protocol for rat treatment in the progression stage.

FIGS. 21A-21E are photos of rat livers at the progression stage of HCC development, showing the results of the direct observation. Panels: A—Rat liver from the negative control group. B—Rat liver from the positive control group with a large tumor. The liver has lost its normal shape. C—Rat pancreas that metastasized. D—Rat lung that metastasized, E—Rat liver from the SIN-treatment group.

FIGS. 22A-22B are bar charts showing the effects of SIN on the weights of rat livers at the progression stage (n=4). The results are expressed as mean±SD, and the data obtained were evaluated by ANOVA. Statistical analyses of the data were performed using the student's t-test. The symbol “*” indicates that p<0.05. Panels: A—Liver weight/body weight index of three groups. B—Liver weight/body weight index of the positive control group and SIN-treatment groups were compared to the negative control group. The negative control group was considered as 100%.

FIG. 23 is a bar chart showing the effects of SIN on the serum ALT and AST levels in rats at the progression stage (n=4). The results are expressed as mean±SD, and the data obtained were evaluated by ANOVA. Statistical analyses of the data were performed using the student's t-test. The symbol “*” indicates that p<0.05. The amount of ALT/AST in rat serum of the positive control and SIN-treatment groups were compared to the negative control group. The negative control group was considered to be 100%.

FIGS. 24A-24D are micrographs of the rat liver sections showing the damaged hepatocyte structure in the liver from the positive control and the restored hepatocyte structure in the liver from the SIN—treatment group at the progression stage. Panels: A—Rat liver section from the negative control group. Clear histological structure of the hepatocyte could be observed. B—Rat liver section from the positive control group. Clusters of fatty droplets presented and cell death surround. C—Rat liver section from the positive control group. Cell size became smaller, and blood vessels and blood increased. D—Rat liver section from SIN-treatment group. The basic structure was restored without abnormal appearance.

FIGS. 25A-25G are micrographs showing the GST-p positive areas of the liver sections at the progression stage. Panels: A—Rat liver section from the negative group (2.5×). No GST-p expression was found in the whole section. B—Rat liver section from the negative group (20×). Normal basic structure could be observed. C—Rat liver section from the positive group (2.5×). GST-p positive area could be found across the section. D—Rat liver section from the positive group (20×). GST-p positive area appeared in clusters, and necrosis cell could be found. E—Rat liver section from the SIN-treatment group (2.5×). Only limited GST-p positive area was found. F—Rat liver section from the SIN-treatment group (20×). The basic structure was restored. G—Rat liver section from the SIN-treatment group (40×), showing an example of the GST-p positive area.

FIG. 26 is a bar chart comparing the percentage of GST-p positive area/whole section area of each group at the progression stage (n=4). The results are expressed as mean±SD, and the data obtained were evaluated by ANOVA. Statistical analyses of the data were performed using the student's t-test. The symbol “*” indicates that p<0.05. GST-p positive area/whole section area ratio of the positive control and SIN-treatment groups are compared with the negative control group.

FIGS. 27A-27D show the mRNA expressions of p. 53 and Mdm2 in rats at the progression stage (n=4). The results are expressed as mean±SD, and the data obtained were evaluated by ANOVA. Statistical analyses of the data were performed using the student's t-test. The symbol “*” indicates that p<0.05. Panels: A—p53 mRNA expression level measured by the ratio of total p. 53 to β-actin. B—p53 mRNA expression measured by RT-PCR. C—Mdm2 expression level measured by the ratio of total Mdm2 to β-actin. D—Mdm2 mRNA expression measured by RT-PCR.

FIGS. 28A-28D show the expressions of total p53 protein and WT p53 protein in rats at the progression stage (n=4). The results are expressed as mean±SD, and the data obtained were evaluated by ANOVA. Statistical analyses of the data were performed using the student's t-test. The symbol “*” indicates that p<0.05. Panels: A—Total p53 expression level measured by the ratio of total p53 to β-actin. B—Total p53 expression measured by Western Blot. C—WT p53 expression level measured by the ratio of WT p53 to β-actin. D—WT p53 expression measured by Western Blot.

FIGS. 29A-29B show the expression of Mdm2 protein in rats at the progression stage (n=4). The results are expressed as mean±SD, and the data obtained were evaluated by ANOVA. Statistical analyses of the data were performed using the student's t-test. The symbol “*” indicates that p<0.05. Panels: A—Mdm2 expression level measured by the ratio of Mdm2 to β-actin. B—Mdm2 protein expression measured by Western Blot.

FIGS. 30A-30D show the expressions of p21 protein and PCNA protein in rats at the progression stage (n=4). The results are expressed as mean±SD, and the data obtained were evaluated by ANOVA. Statistical analyses of the data were performed using the student's t-test. The symbol “*” indicates that p<0.05. Panels: A—p21 expression level measured by the ratio of p21 to β-actin. B—p21 protein expression measured by Western Blot. C—PCNA expression level measured by the ratio of PCNA to β-actin. D—PCNA protein expression measured by Western Blot.

FIGS. 31A-31D show the expressions of Bax protein and Bcl-2 protein in rats at progression stage (n=4). The results are expressed as mean±SD, and the data obtained were evaluated by ANOVA. Statistical analyses of the data were performed using the student's t-test. The symbol “*” indicates that p<0.05. Panels: A—Bax expression level measured by the ratio of Bax to β-actin. B—Bax protein expression measured by Western Blot. C—Bcl-2 expression level measured by the ratio of Bcl-2 to β-actin. D—Bcl-2 protein expression measured by Western Blot.

### DETAILED DESCRIPTION OF THE INVENTION

The present invention provides a pharmaceutical composition for treating cancer in a subject, comprising sinigrin or a pharmaceutically acceptable derivate thereof, or a mixture of both, and a pharmaceutically acceptable carrier.

In embodiments of the invention, the composition comprises a therapeutically effective amount of sinigrin or a pharmaceutically acceptable derivate thereof or a mixture of both.

Sinigrin of the invention is a unique pure compound with a low molecular weight of 397.46, and a chemical formula of C₁₀H₁₆NO₉S₂K, which is preferably obtained from seeds of Brassica nigra (common name Black mustard). Preferably, sinigrin has the following chemical structure:

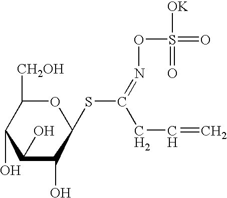

In certain embodiments, sinigrin or a pharmaceutically acceptable derivate thereof or a mixture of both is the major active component. In more preferred embodiments, sinigrin or a pharmaceutically acceptable derivate thereof or a mixture of both is the only active component, as in the case of sinigrin aqueous solution.

Sinigrin or a pharmaceutically acceptable derivate thereof or a mixture of both can be incorporated into the composition of the invention in the form of hydrate. In one embodiment, sinigrin is in the form of sinigrin monohydrate.

The term “effective compound” or “effective ingredient” or “effective component” as used herein refers to sinigrin or a derivate thereof or a mixture of both that is mentioned above. In one embodiment, the sinigrin is extracted from the seeds of Brassica nigra.

The term “therapeutically effective amount” or “effective amount” as used herein is intended to mean an amount of the active component effective to achieve its intended purposes, such as treating cancer.

Normally an effective amount of the active component ranges from bout 0.1 to 300 mg per kilogram body weight, more preferably from about 1 to 100 mg per kilogram body weight, per day, ordinarily in one to four portions. However, in most instances, an effective daily amount will be in the range of from about 1 mg/kg to about 25 mg/kg of body weight, and in another embodiment is about 10 mg/kg of body weight, administered in single or divided doses. In some cases, however, it may be necessary to use dosages outside these limits, which can easily be determined by the prescribing physician or another individual of ordinary skill in the art.

Pharmaceutical compositions may comprise at least one active compound in a pharmaceutically acceptable form, i.e. sinigrin or a pharmaceutically acceptable derivate thereof or a mixture of both, optionally combined with a pharmaceutically acceptable carrier.

The term “pharmaceutically acceptable carrier” as used herein refers to excipients and auxiliaries that facilitate processing of the active component of the invention into formulations that can be used pharmaceutically. The formulations of the pharmaceutical composition can be administered by any desired route of administration, including orally, topically, intramuscularly, intraperitoneally, subcutaneously, intratumorally or intravenously.

The formulations of the pharmaceutical composition described herein, particularly those such as tablets, dragees, troches and capsules, as well as suitable solutions, may contain from about 0.01 to 99.99 percent by weight, or from about 25 to 75 percent by weight of active component(s) together with the excipient and/or auxiliary.

Suitable excipients used in the invention include fillers such as saccharides (e.g., lactose sucrose, mannitol, sorbitol); cellulose derivatives; magnesium sulfate; calcium phosphates (e.g., tricalcium phosphate, calcium hydrogen phosphate); binders such as starch paste (e.g., maize starch, wheat starch, rice starch, potato starch), gelatin, tragacanth, and/or polyvinylpyrrolidone.

Suitable auxiliaries that may be used in the invention include flow-regulating agents and lubricants, such as talc, silica, stearic acid or derivates thereof (e.g., magnesium stearate), and/or polyethylene glycol. Dragee cores are provided with suitable coatings that, if desired, are resistant to gastric juices. For this purpose, concentrated saccharide solutions optionally containing gum arabic, talc, polyvinyl pyrrolidione, polyethylene glycol and/or titanium dioxide; lacquer solutions; or suitable organic solvents or solvent mixtures can be used. In order to produce coatings resistant to gastric juices, i.e., enteric coatings, solutions of suitable cellulose preparations such as acetylcellulose phthalate or hydroxypropylmethyl cellulose phthalate are used. Dyestuffs or pigments can be added to the tablets or the dragee coatings.

The composition of the present invention may be formulated in the form of injections, such as intravenous, subcutaneous, and intramuscular injections, suppositories, or sublingual tablets. In one embodiment, the composition is formulated in an oral administration form.

Alternatively, one may administer the composition in a local, for example, via injection of the compound directly into a tumor, i.e. intratumorally, often in a depot or sustained release formulation. In the embodiment described herein, a variety of delivery systems for sinigrin or the pharmaceutical composition may be employed, including, but not limited to, liposomes and emulsions. Furthermore, one may administer the agent in a targeted drug delivery system, for example, in a liposome coated with a tumor-specific antibody. The liposomes will then be targeted to, and taken up selectively by, the tumor.

Pharmaceutical formulations in the dosage form of, e.g., injections, suppositories, sublingual tablets, tablets, and capsules are prepared according to methods commonly accepted in the art.

In preparing injections, the effective ingredient is blended, if necessary, with a pH modifier, a buffer, a solubilizing agent, a suspending agent, a stabilizer, and a preservative, followed by preparation of an intravenous, subcutaneous, or intramuscular injection according to an ordinary method.

Examples of the solubilizing agent include polyoxyethylene hydrogenated castor oil, polysorbate 80, nicotinamide, polyoxyethylene sorbitan monolaurate, macrogol, and an ethyl ester of castor oil fatty acid. Examples of the suspending agents include methylcellulose, polysorbate 80, hydroxyethylcellulose, acacia, powdered tragacanth, sodium carboxymethylcellulose, and polyoxyethylene sorbitan monolaurate.

Stabilizers include sodium sulfite, sodium metasulfite, and ether. Preservatives include methyl p-hydroxybenzoate, ethyl p-hydroxybenzoate, sorbic acid, phenol, cresol, and chlorocresol.

When the active compound or the composition is administered orally, it can be in the form of tablets or capsules, or as an aqueous solution or suspension.

In the case of tablets, commonly used carriers include lactose, mannitol and corn starch. Also, lubricating agents, such as magnesium stearate, are commonly added. In the case of the capsule form, the active compound can be administered in dry form in a hard gelatin capsule or in a suitable gelled or liquid vehicle, such as a liquid polyethylene glycol or a carrageenan gel, in a soft gelatin capsule.

When liquid solutions are required for oral use, sinigrin or a pharmaceutically acceptable derivate thereof or a mixture of both is dissolved in diluents such as saline, water or polyethylene glycol (e.g., PEG 400). In one embodiment, the active ingredient is dissolved in water. For oral aqueous suspensions, the active ingredient may be combined with emulsifying and suspending agents. If desired, certain sweetening and/or flavoring agents may be added.

In embodiments of the invention, the cancers to be treated include liver cancer, pancreatic cancer and lung cancer. In one embodiment, the cancer is liver cancer.

In some embodiments, the cancers to be treated may be primary or secondary, at a promotion stage or at a progression stage. The promotion stage means that the subjects to be treated are at risk of cancer but show no symptoms of cancer yet. For example, the subjects can be those exposed to large amounts of radiation or carcinogens. The progression stage means that tumors are already formed, such as the tumors in diagnosed patients.

In other embodiments, the cancers have already metastasized. Examples include, but are not limited to, cancers metastasized from liver to lung or from liver to pancreas, which are known as secondary cancers.

In some embodiments where the cancers are at the progression stage, the composition not only eliminates or minimizes the primary tumors, but also suppresses metastasis of tumor cells.

In some embodiments of the pharmaceutical composition described herein, the subject being treated with the active compound or the composition is a mammal. In one embodiment, the subject is a human patient.

The pharmaceutical composition or the active ingredient described herein is capable of reducing cell viability of tumor cells, such as human hepatoma cells, while causing no harm to healthy cells. The compositions or active ingredient described herein may inhibit the proliferation of tumor cells through a G0/G-1 phase arrest mechanism. In certain embodiments, the pharmaceutical composition or the active ingredient of the present invention reduces cell viability of tumor cells to about 90%, about 80%, about 70%, about 60%, about 50%, about 40% or less than about 40%.

The composition or the active ingredient described herein is also capable of inducing apoptosis of tumor cells such as human hepatoma cells. In some embodiments, the occurrence of apoptosis of the tumor cells can be verified by an observable DNA ladder. In some embodiments, mRNA expression of genes in drug toxicity and metabolism pathways is changed in response to the composition or the active ingredient. Such genes include, but are not limited to, cyp4b1, cyp4f3, cyp7a1, por, nat2, nat5, nat8, mgst1, arnt, xrcc2, nudt1, rad50, rad51, cdkn1a, tnf, tnfrsf11a, bcl-2, rad23a, chek2, dpyd, ccng, atm, fgf2, rarb, cct2, cct4, cct5, and ar. The expression change can be an increase or a decrease. Changes in expression of these genes can range from 3 fold to 7 fold, or higher.

In other embodiments, administration of the composition or the active ingredient results in expression changes of genes related to apoptosis and oncogenesis at mRNA or protein level or both, no matter the stage of the cancer (i.e. the cancer can be at the promotion stage or at the progression stage). Such genes include p. 53, mdm2, p. 21, pcna, bax and bcl-2. Administration of the composition or the active ingredient reverses the enhanced expressions of p53 (total), Bcl-2, Mdm2 and PCNA and restores the decreased expressions of p. 53 (wild type), p21 and Bax in tumors.

The methods described herein also relate to a method of treating cancer with sinigrin or a pharmaceutical composition described herein. The method, for example, can comprise a step of administering a therapeutically effective amount of active components of the invention such as sinigrin, or a pharmaceutically acceptable derivate thereof or a mixture of both, or a pharmaceutical composition of the invention to a subject at risk of or suffering from cancer.

The active components or the composition of the present invention may be administered intravenously, subcutaneously, and intramuscularly, sublingually, orally, locally or intratumorally, employing a variety of dosage unit forms which comprise a dosage unit of sinigrin or a pharmaceutically acceptable derivate thereof or a mixture of both. The dosage unit of sinigrin or a pharmaceutically acceptable derivate thereof or a mixture of both is in the range of about 0.1 mg to about 100 mg, about 1 mg to about 15 mg, or may be 1 mg, 2.5 mg, 5 mg or 10 mg.

The dose or effective amount will vary depending upon the symptoms and severity of the cancer, sex, age, and weight of patients, method of administration, time and intervals of administration and properties, dispensing, and kind of pharmaceutical formulations, specific effective ingredients, etc. It is appreciated for those skilled in the art that there is no particular limitation with respect to the dose. Normally the active component may be administered in an effective amount of about 0.1 to 300 mg per kilogram body weight, or 1 to 100 mg per kilogram body weight, per day, ordinarily in one to four portions. However, in most instances, an effective daily amount will be in the range of from about 1 mg/kg to about 25 mg/kg of body weight, and may be about 10 mg/kg of body weight, administered in single or divided doses. In some cases, however, it may be necessary to use dosages outside these limits, which will be determined by the prescribing physician. A variety of techniques for formulation and administration may be found in Remington's Pharmaceutical Sciences, 18th ed., Mack Publishing Co., Easton, Pa. (1990).

Certain embodiments described herein relate to a method of inhibiting growth of hepatoma cells comprising contact the hepatoma cells with a pharmaceutical composition described herein or sinigrin or a pharmaceutically acceptable derivate thereof or a mixture of both.

Other embodiments relate to a method of inducing apoptosis of hepatoma cells comprising contact the hepatoma cells with a pharmaceutical composition described herein or sinigrin or a pharmaceutically acceptable derivate thereof or a mixture of both.

In some embodiments, the hepatoma cells are mammalian cells, including human hepatoma cells.

Certain embodiments also relate to the use of sinigrin or a pharmaceutically acceptable derivate thereof or a mixture of both in manufacturing a medicament or a pharmaceutical composition provided herein for treatment of cancer in a subject. The medicament or pharmaceutical composition can be administered to the subject at risk of or suffering from the cancers described herein.

The pharmaceutical composition may be prepared by conventional methods, such as a variety of techniques for formulation found in Remington's Pharmaceutical Sciences, 18th ed., Mack Publishing Co., Easton, Pa. (1990).

### EXAMPLES

The present embodiments are further illustrated by the following series of examples. The examples are provided for illustration and are not to be construed as limiting the scope or content of the invention in any way.

### Example 1

Cell Viability Assay

The neutral red (NR) cytotoxicity assay is a chemosensitivity assay for measuring cell survival/viability, based on the ability of viable cells to incorporate and bind neutral red, a supravital dye. NR is a weak cationic dye that readily penetrates cell membranes by non-ionic diffusion, accumulating intracellularly in lysosomes, where it binds with anionic sites in the lysosomal matrix. Alterations of the cell surface or the sensitive lysosomal membrane lead to lysosomal fragility and other changes that gradually become irreversible. Such changes brought about by the action of xenobiotics result in a decreased uptake and binding of NR. It is thus possible to distinguish between viable, damaged, or dead cells. (Babich, H et al, 1990)

Sinigrin Monohydrate was purchased from Fluka. The Neutral Red dye powder, Na₂HPO₄, NaH₂PO₄ and NaHCO₃ were purchased from Sigma. NaCl and SDS powders were purchased from USB. Trypsin, Fetal Bovine Serum, PSN antibiotics mix, the cell medium DMEM and RPMI powders were purchased from GibcoBRL, USA. The 96-well plate was purchased from IWAKI, JP. Reagents used are listed in Table 1 below:

TABLE 1
Reagents Used in Cell Viability Assay

Sinigrin Stock Solution

| | |
|---|---|
| Sinigrin monohydrate | 1 g/5 ml |
| ddH₂O | 5 ml/5 ml |

The above solution was well mixed and aliquoted into microcentrifuge
tubes (0.5 ml each). The stock solution was diluted with the medium
and filtered with 0.22 μm filters before use.
PBS (10X)

| | |
|---|---|
| Na₂HPO₄ | 10.9 g/1 L |
| NaH₂PO₄ | 3.2 g/1 L |
| NaCl | 90 g/1 L |

The solution was made up to 1 L with ddH₂O.
PBS (1X) (pH = 7.4)

| | |
|---|---|
| 10X PBS | 100 ml/1 L |

The solution was made up to 1 L with ddH₂O, and pH was adjusted to 7.4.
Neutral Red Dye

| | |
|---|---|
| Neutral Red Dye | 0.4 g/50 ml |

The dye was made up to 50 ml with PBS. After complete dissolution, the
dye was filtered before use.
DMEM/RPMI Plain Medium (pH 7.4)

| | |
|---|---|
| DMEM/RPMI Medium powder | 48.6 g (3 bags)/3 L |
| NaHCO₃ | 11.1 g/3 L |

The solution was made up to 3 L with ddH₂O and filtered in a culture
hood before use. pH of the solution was adjusted to 7.4.
DMEM/RPMI Complete Medium

| | |
|---|---|
| DMEM/RPMI plain medium | 450 ml/500 ml |
| Fetal Bovine Serum | 50 ml/500 ml |
| PSN | 5 ml/500 ml |

The complete medium was mixed before use.

Three different cell lines HepG2, WRL-68 and Clone 9 cell lines were used in the experiment. HepG2 is a human hepatoblastoma cell line. It is a well known cell model in anti-HCC studies (Alshatwi A A, et al, 2006). WRL-68 is a human normal liver embryo cell line that has a morphologic structure similar to hepatocytes and hepatic primary cultures (Gutierrez-Ruiz, et al, 1994). This cell line was used as the control. Clone 9 is the rat Sprague Dawley (SD) liver normal cell line. In the in vivo studies, sinigrin (SIN) would be applied to the SD rat animal model. The employment of this cell line in the cell viability study was to test if SIN would be toxic in the rat animal model.

HepG2, WRL-68 and Clone 9 cells were grown in complete culture medium (RPMI or DMEM), trypsinized and washed. Ten thousand cells of each cell type were seeded in 96-well plates. After 24 hours of pre-incubation, cells were treated with different concentrations of SIN and incubated for 72 hours. After incubation, cells were harvested and washed twice with 1×PBS buffer. Fifty microliters of Neutral Red solution were added to each well. The whole plate was placed into an incubator at 37° C. with 5% CO₂. After incubation for 1 hour, the plate was washed twice with 1×PBS buffer, and completely dried in a 60° C. oven overnight. 100 μl of 1% SDS solution was added to each well to lyse the cells and resolve the Neutral Red dye. The color was measured at OD₅₄₀ₙₘ.

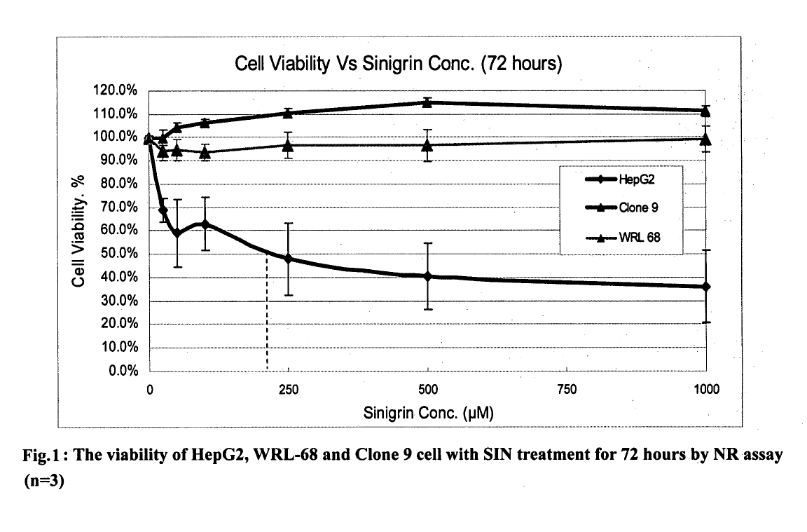

FIG. 1 shows the viability of HepG2, WRL 68 and HepG2 cells after treated with SIN of different concentrations for 72 hours. Even at a low concentration (about 25 μM), SIN was able to reduce the viability of HepG2 cells to about 70%. With the SIN concentration increasing to 1,000 μM, viability of HepG2 cells continuously decreased to about 35%. IC₅₀ value for HepG2 cells was lower than 250 μM. In contrast, no decrease in cell viability was observed in cultures of healthy liver cells (WRL-68 cells or Clone 9 cells) even the SIN concentration has reached 1,000 μM. The results indicate that SIN is capable of specifically inhibiting the growth of tumor cells.

### Example 2

Cell Cycle Analysis

Flow Cytometery is a rapid and quantitative method for measuring certain physical and chemical characteristics of cells or particles as they travel in suspension through a sensor. When the cell is labeled with propidium iodide (PI), the DNA content can be measured which is used to determine the stage of the cell cycle. When cell cycles are obtained, a cell cycle map can be recorded.

Sheath Fluid was purchased from FACSFlow™ and is ready for use. Ethanol was purchased from BDH. PI was purchased from Sigma. RNase A was purchased from USB. Reagents used are listed in Table 2 below:

TABLE 2
Reagents Used in Cell Cycle Analysis
PI Solution

| | |
|---|---|
| PI | 0.4 mg/10 ml |
| RNase A | 1 mg/10 ml |
| 1X PBS | 10 ml/10 ml |

The solution was stored in the dark at −20° C.

HepG2 cells were trypsinized, washed and seeded into 25 mm² culture plates in complete RPMI medium. After 24 hours of pre-incubation, different concentrations (250 μM and 500 μM) of SIN were added to the culture plates. The complete RPMI medium was added to one plate containing control cells. After different periods of incubation with SIN, cells were harvested, the medium was removed from the culture plate and the plate was washed twice with 2 ml of 1×PBS buffer. All of the solutions were collected, and the cells were trypsinized and collected. The whole solution was centrifuged at 1,000 rpm for 3 minutes. The supernatant was removed and the cell pellet was resuspended in 1 ml 1×PBS buffer for washing. The suspension was transferred to a microcentrifuge tube. The washing solution was centrifuged at 1,000 rpm for 3 minutes, and the supernatant was discarded.

The cell pellet was resuspended in 1 ml of 70% ethanol and 0.1 ml of 1×PBS buffer. The suspension was kept at 4° C. overnight to fix the cells. After centrifugation at 1,000 rpm for 3 minutes, 1 ml of 1×PBS was added for washing. PI solution (1 ml) was added to the cells which were then incubated at 37° C. for 30 minutes. DNA content was analyzed using an FACScan Flow cytometry with sufficient Sheath Fluid. The results were analyzed by the FCS express software produced by the De Novo Software Company.

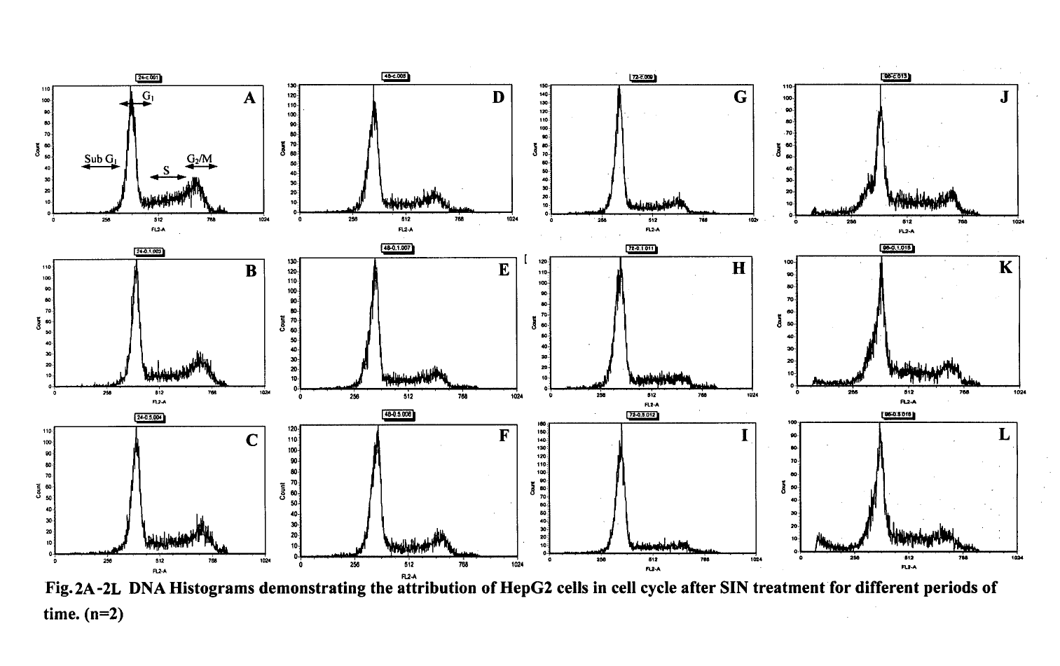

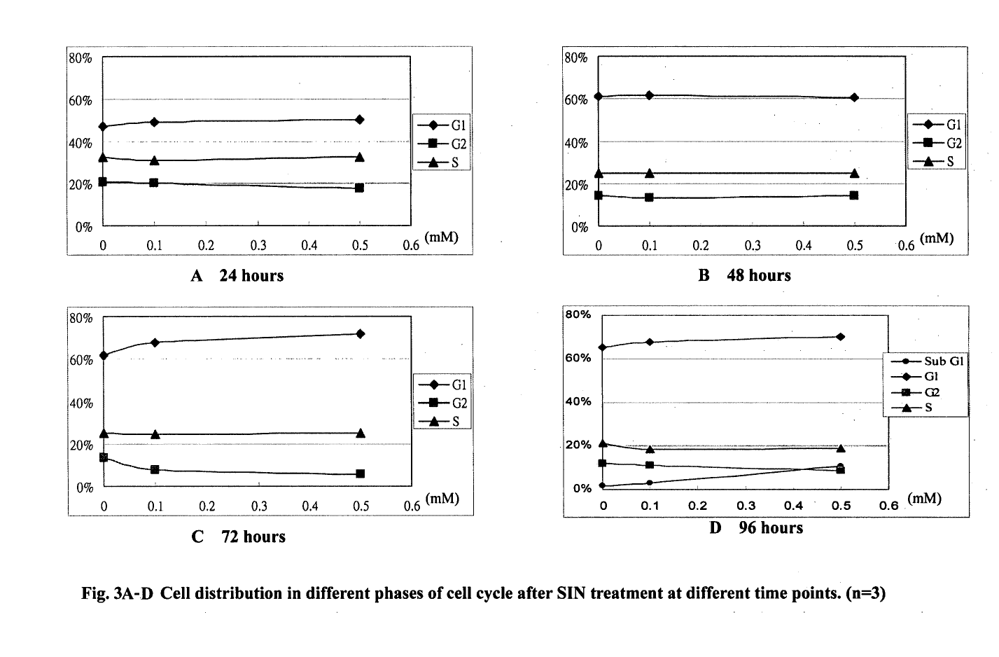

SIN treatment results in G0/G1 phase arrest in HepG2 cells (FIGS. 2, 3). After treating HepG2 cells with 0.5 mM SIN for 96 hours, the increase of the cells arrested in sub G1 phase becomes very obvious as compared with that of the untreated cells (FIGS. 2J, L). FIG. 3 further demonstrates the arrested sub-G1 phase by showing the cell distribution in different phases of the cell cycle after SIN treatment at different concentrations at different time points. The number of cells at phases subsequent to G1 phase (S and G2) decreased, while those of the cells at G1 and sub G1 phases increased (FIG. 3). The above results suggest that growth of SIN treated HepG2 cells is inhibited by a G0/G1 phase arrest mechanism.

### Example 3

### Apoptosis Determination by DNA Fragmentation Assay

DNA fragmentation or DNA laddering is an indication of apoptosis. During the apoptosis process, the enzymes involved in DNA repair and cell replication are inactivated and nuclear proteins are degraded. After the nuclear structure is fragmented, DNA inside the nucleus is fragmented by the enzyme Caspase Activated DNase. This enzyme is only activated during apoptosis and causes the fragmentation of DNA into nucleosomal units (Hugh J M Brady, 2004). DNA fragmentation is used to identify the apoptosis event.

EDTA, Glycerol, Xylene Cyanole, RNase A and Protease K were purchased from Sigma. Bromophenol Blue, Agarose, Tris-Base, Boric Acid, NaCl and SDS powder were purchased from USB. Ethanol was purchased from BDH. Reagents used are listed in Table 3 below:

TABLE 3
Reagents Used in DNA Fragmentation Assay

Lysis Buffer (pH 8.3)

| | |
|---|---|
| Tris base | 1.2114 g/50 ml |
| 0.5M EDTA (pH 8.0) | 10 ml/50 ml |
| 10% SDS | 5 ml/50 ml |

The solution was made up to 50 ml by ddH₂O and the pH of the solution
was adjusted to 8.3.
TE Buffer with RNase A (pH 8.0)

| | |
|---|---|
| Tris Base | 121.14 mg/10 ml |
| 0.5M EDTA (pH 8.0) | 20 μl/10 ml |
| RNase A | 2 mg/10 ml |

The solution was made up to 10 ml by ddH₂O and the pH of the solution
was adjusted to 8.0.
1X TBE Buffer

| | |
|---|---|
| Tris Base | 10.8 ml/1 L |
| Boric Acid | 5.5 ml/1 L |
| 0.5M EDTA (pH 8.0) | 20 ml/1 L |

The solution was made up to 1 L by ddH₂O.
6X DNA loading Dye

| | |
|---|---|
| Glycerol | 93.6 μl/250 μl |
| 0.5M EDTA (pH 8.0) | 3 μl/250 μl |
| Bromophenol Blue | 0.3 mg/250 μl |
| Xylene Cyanole | 0.3 mg/250 μl |
| ddH₂O | 153.4 μl/250 μl |

EB solution (0.5 mg/ml)

| | |
|---|---|
| EB powder | 50 mg/100 ml |
| ddH₂O | 100 ml/100 ml |

HepG2 cells were treated and harvested as described in the foregoing examples. The cell pellet was re-suspended in 400 μl of the lysis buffer by vortexing. Twenty microliters of 10 mg/ml protease K were added into the solution after a complete dissolution. The solution was incubated at 37° C. for 3 hours to completely lyse the cells. After incubation, the solution was allowed to cool to room temperature. One hundred and fifty microliters of a saturated NaCl solution were added into the cell lysate and vortexed. The lysate was centrifuged at 7,000 rpm for 15 minutes. The supernatant was collected in a new microcentrifuge tube. One milliliter of ice cold absolute ethanol was added to the solution to precipitate DNA. After precipitation, the microcentrifuge tube was centrifuged at 14,000 rpm for 20 minutes at 4° C. The supernatant was removed and the DNA pellet was washed with 70% ethanol and centrifuged again. The pellet was allowed to dry at room temperature. Fifty microliters of TE buffer containing RNase A were added into the microcentrifuge tube to dissolve DNA after drying. And the samples were allowed to incubate at 37° C. for 2 hours for dissolving DNA.

0.3 g agarose, 20 ml TBE buffer and 3 μl EB were mixed and heated to make a 1.5% agarose gel for DNA fragmentation assay. Ten microliters of the dissolved sample were mixed with 2 μl of 6×DNA loading dye to make the loading sample. The sample was run on 1.5% agarose gel at 80V for 1 hour. The DNA bands were examined under a UV illuminator (UVP) and the gel was photographed for documentation.

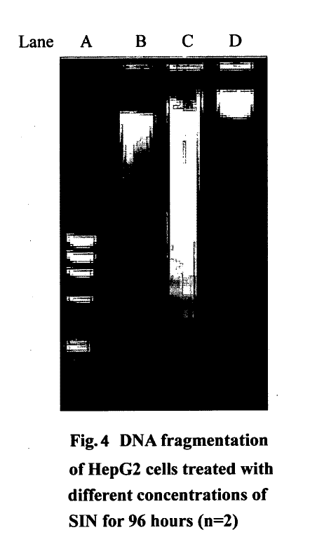

A DNA ladder started to appear on the upper part of the agarose gel in the HepG2 cells treated with 0.25 mM SIN (FIG. 4 lane B). In the HepG2 cells treated 0.5 mM SIN for 96 hours, a DNA ladder was clearly visible, which indicated that these cells underwent apoptosis (FIG. 4 lane C). No DNA cleavage or degradation was observed in untreated HepG2 cells (FIG. 4 lane D).

### Example 4

### cDNA Microarray

The advancement of nucleic acid array technology has made it possible to analyze the expression of multiple genes in a single experiment. The cDNA Microarray is used to characterize gene expression associated with a specific biological pathway in a more comprehensive and cost effective manner. The genes on the cDNA microarray membrane are targeted to the drug toxicity and metabolism pathways. It is a useful approach to study the biological activities of SIN.

Oligo GEArray Human Drug Metabolism and Toxicology microarray membrane (OHS-401) and Oligo GEArray Reagent Kit with Truelabeling-AMP2.0 (GA-034) were purchased from SuperArray. Biotin-UTP was purchased from ROCHE. Diethyl pyrocarbonate (DEPC), MOPS, SDS, NaCl, Sodium Citrate dehydrate, Sodium acetate and Agarose were purchased from USB. Chloroform, isopropanol, and formaldehyde (12.3M) were purchased from Sigma. TRIzol® reagent was purchased from Invitrogen. Ethanol was purchased from BDH. Super RX X-Ray Film was purchased from FujiFilm Ltd. Contents of the Oligo GEArray Reagent Kit are listed in Table 4 below, and other used reagents are listed in Table 5 below:

TABLE 4
Contents of Oligo GEArray Reagent Kit
Oligo GEArray Reagent Kit with
Truelabeling-AMP2.0 (GA-034) contains:

| | |
|---|---|
| G1 | TreuLabeling Primer |
| RI | RNase Inhibitor |
| G2 | cDNA Synthesis Enzyme Mix |
| G3 | 5X cDNA Synthesis Buffer |
| G24 | 2.5 X RNA Polymerase Buffer |
| G25 | RNA Polymerase Enzyme |
| G6 | Lysis & Binding Buffer |
| G17 | Washing Buffer |
| G26 | RNase-Free 10 mM Tris buffer (pH 8.0) |
| | Spin Columns, Elution tubes |
| | GEAhyb Hybridization Solution |
| | GEAblocking Solution Q |
| | 5X Buffer F |
| | AP-SA (undiluted) |
| | Buffer G |
| | CDP-Star chemiluminescent substrate |

TABLE 5
Other Reagents Used in cDNA Microarray:

10X MOPS

| | |
|---|---|
| MOPS | 83.72 g/1 L |
| NaOAC | 8.203 g/1 L |
| EDTA | 3.722 g/1 L |

The solution was made up to 1 L by autoclaved DEPC-H₂O.
RNA Agarose Gel

| | |
|---|---|
| Agarose | 0.3 g/20 ml |
| Autoclaved DEPC-H₂O | 14.4 ml/20 ml |
| 10X MOPS | 2 ml/20 ml |
| Formaldehyde (12.3 M) | 3.6 ml/20 ml |

The solution was heated and cooled down in a gel mold with comb.
Autoclaved DEPC-H₂O

| | |
|---|---|
| DEPC | 1 ml/2 L |
| ddH₂O | 1999 ml/2 L |

The solution was well mixed and autoclaved before use.
RNA Sample Buffer

| | |
|---|---|
| Formamide | 10 ml/15.5 ml |
| Formaldehyde | 3.5 ml/15.5 ml |
| 10X MOPS buffer | 1 ml/15.5 ml |
| Autoclaved DEPC-H₂O | 1 ml/15.5 ml |

6X RNA loading Dye

| | |
|---|---|
| Glycerol | 250 μl/500 μl |
| 0.5M EDTA (pH 8.0) | 1 μl/500 μl |
| Bromophenol Blue | 0.3 mg/500 μl |
| ddH₂O | 153.4 μl/500 μl |

Washing Solution 1

| | |
|---|---|
| SDS | 1 g/100 ml |
| NaCl | 1.753 g/100 ml |
| Sodium Citrate Dihydrate | 0.882 g/100 ml |

The solution was made up to 100 ml by autoclaved DEPC-H₂O..
Washing Solution 2

| | |
|---|---|
| SDS | 0.5 g/100 ml |
| NaCl | 87.65 mg/100 ml |
| Sodium Citrate Dihydrate | 44.1 mg/100 ml |

The solution was made up to 100 ml by autoclaved DEPC-H₂O.
1X Buffer F

| | |
|---|---|
| 5X Buffer F | 4 ml/20 ml |
| Autoclaved DEPC-H₂O | 16 ml/20 ml |

Diluted AP-SA (1:8,000)

| | |
|---|---|
| AP-SA (undiluted) | 1 μl/8 ml |
| 1X Buffer F | 8 ml/8 ml |

HepG2 cells were treated and harvested as described in the foregoing examples. The cell pellet was dissolved in 500 μl TRIzol® reagent (Invitrogen, CA, USA). The whole solution was allowed to stand at room temperature for 5 minutes. The solution was centrifuged at 14,000 rpm for 10 minutes at 4° C. The supernatant was collected in a new tube with 100 μl chloroform. After shaking and incubation for 10 minutes at room temperature, the solution was centrifuged at 14,000 rpm for 15 minutes at 4° C. Recentrifugation was necessary if the layer was not clear. The upper aqueous layer was carefully transferred into a new tube with 250 μl isopropanol. After shaking, the tube was allowed to stand at room temperature for 10 minutes for RNA precipitation. The tube was centrifuged at 14,000 rpm for 10 minutes at 4° C. The supernatant was discarded and 0.5 ml of 75% ethanol was added into the tube for washing. The tube was centrifuged at 7,500 rpm for 5 minutes at 4° C. The supernatant was carefully pipetted off and the pellet was allowed to dry at room temperature. Fifty microliters of autoclaved DEPC—H₂O were added to dissolve the RNA at 55° C. for 15 minutes.

The RNA samples were determined by spectrophotometery at OD 280 nm, 260 nm and 320 nm. The RNA samples were 1000× diluted with 1×TE buffer (pH 8.0). 1×TE buffer was used as the blank in spectrophotometery. The quality of RNA samples was acceptable when the reading ratio at OD 260 nm and OD 280 nm was greater than 2.0. The quantity of RNA in the samples were calculated with the formula (one unit absorbance at OD 260 nm=40 μg/ml standard RNA) and corrected with dilution factor.

Reverse Transcription of mRNA to cDNA was performed by Superarray TrueLabeling-AMP™ 2.0 kit. 3 micrograms of total RNA, 1 μl of oligo dT primer (G1) and certain volume of RNase-Free H₂O were mixed to a total volume of 10 μl. The mixture was incubated at 70° C. for 10 minutes and chilled onto ice immediately. Four microliters of RNase-Free H₂O, 4 μl of 5×cDNA synthesis Buffer (G3), 1 μl of RNase Inhibitor, and 1 μl of cDNA Synthesis Enzyme Mix (G2) were added to each tube and mixed. The tubes were incubated at 42° C. for 50 minutes followed by 75° C. for 5 minutes and cooled down to 37° C.

Three micrograms of RNA of each sample were used for RNA gel electrophoresis. Various volumes of RNA samples and 2 μl of RNA loading dye were mixed and RNA loading buffer was added to make the total mix volume of 20 μl. The loading mixture was incubated at 70° C. for 15 minutes to denature the RNA.

The RNA gel was prepared as described in Table 5 and 1×MOPS was used as the running buffer. Twenty microliters of RNA samples loading mixtures were applied to the gel and run for 35 minutes at 100V. The gel was post-stained with EB for certain time and de-stained with Autoclaved DECP-water. The RNA bands were examined under a UV illuminator (UVP) and the gel was photographed for documentation. Sharp bands for the 28s and 18s ribosomal RNA were considered as good quality RNA.

Sixteen microliters of 2.5×RNA Polymerase Buffer (G24), 2 μl of Biotinylated-UTP (10 mM), and 2 μl of RNA Polymerase Enzyme (G25) were added to each tube and mixed. The whole mixture was incubated for one hour at 37° C.

cRNA purification was performed with SuperArray Array Grade cRNA Cleanup Kit. 60 RNase-Free H₂O was added into each cRNA synthesis reaction tube for a final volume of 100 μl. The entire reaction mixture was transferred to one 1.5-ml RNase-Free tube. Three hundred and fifty microliters of Lysis & Binding Buffer (G6) were added to each reaction mixture and mixed. After mixing with 350 μl of 100% ethanol, each sample was immediately loaded onto the center of its own Spin Column. The spin column was then centrifuged for 30 seconds at 8,000×g. The flow-through was discarded and 600 μl Washing Buffer (G17 with ethanol) was added to each spin column. The spin column was centrifuged for 30 seconds at 8,000×g. The flow-through was discarded and 200 μl Washing Buffer (G17 with ethanol) was added to each spin column. The spin column was centrifuged for 3 minutes at 11,000×g, transferred to a fresh elution tube and the flow-through was discarded. Fifty microliters of RNase-Free 10 mM Tris Buffer (pH 8.0) (G26) were added to the center of each spin column. The column was allowed to incubate at room temperature for 2 minutes and centrifuged for 1 minute at 8,000×g. The flow-through was the purified cRNA. The quality and quantity of the cRNA product were determined as described previously.

Oligo GEArray® Microarray Human Drug Metabolism and Toxicity membranes were used. The array membranes were pre-wetted with 5 ml deionized water. The hybridization tubes were allowed to sit inverted for 5 minutes to test for leakage. The GEAhyb Hybridization Solution was warmed to 60° C. before use. The water in the hybridization tubes was discarded and 2 ml of hybridization solution was added to each tube for pre-hybridization at 60° C. After 2 hours pre-hybridization, original pre-hybridization solution was discarded and 2 μg of cRNA product with 0.75 ml of hybridization solution was added into each tube and allowed to hybridize overnight at 60° C.

After hybridization, the hybridization mix was poured to a new clear microcentrifuge tube and stored at −20° C. Five milliliters of Washing Solution 1 (1% SDS, 0.3M NaCl, 0.03M sodium citrate dihydrate) were added to the hybridization tube. The hybridization tube was placed in a 60° C. oven and washed for 15 minutes at 25 rpm. Washing Solution 1 was discarded after washing and 5 ml of Washing Solution 2 was added to the hybridization tubes. The sample was placed in the 60° C. oven and washed for exactly 15 minutes at 25 rpm. The washing solution was immediately discarded. The hybridization tubes and oven were allowed to cool to room temperature. Two milliliters of GEAblocking Solution Q were added to each hybridization tube and vortexed. The hybridization tube was placed in the room temperature oven and incubated for 40 minutes at 25 rpm. After incubation, Solution Q was discarded and 2 ml of Dilute AP-SA Buffer were added to the hybridization tube and allowed to incubate for exactly 10 minutes with continuous 7.5 rpm agitation. The membranes were washed four times with 4 ml of 1× Buffer F for 5 minutes with gentle agitation. After the last wash, the membranes were rinsed twice with 3 ml of Buffer G. One milliliter of a CDP-star chemiluminescent substrate was added into each hybridization tube and incubated for 5 minutes. The membrane was wrapped in Saran foil and exposed to an X-ray film immediately for different time.

The films which captured the signal from the ECL reaction were scanned by a computer and analyzed by the online software provided by the SuperArray website. The background setting was “minimum value” and the density setting was “average”.

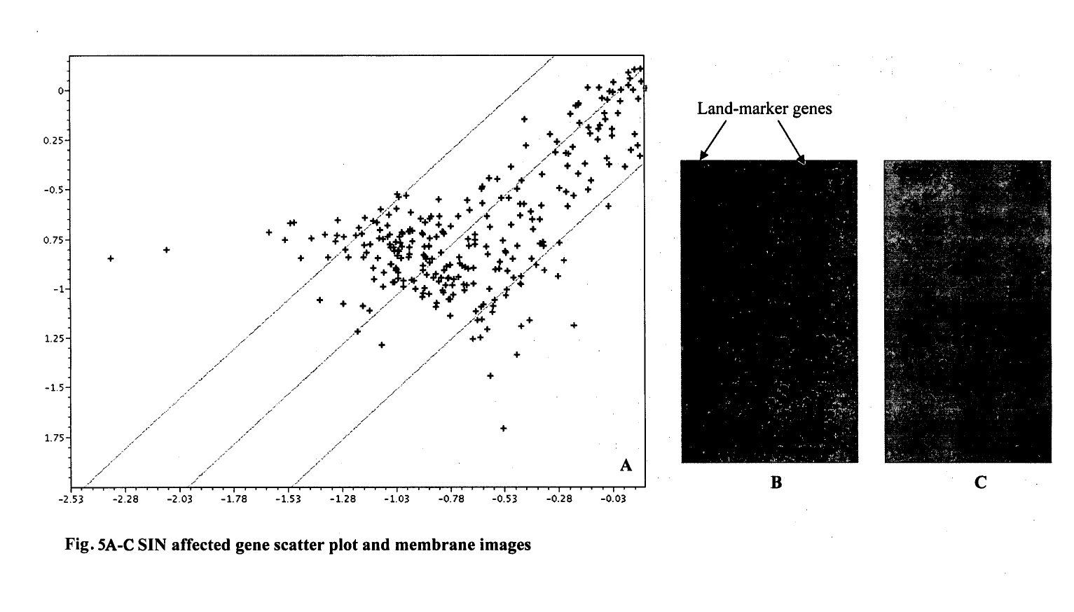

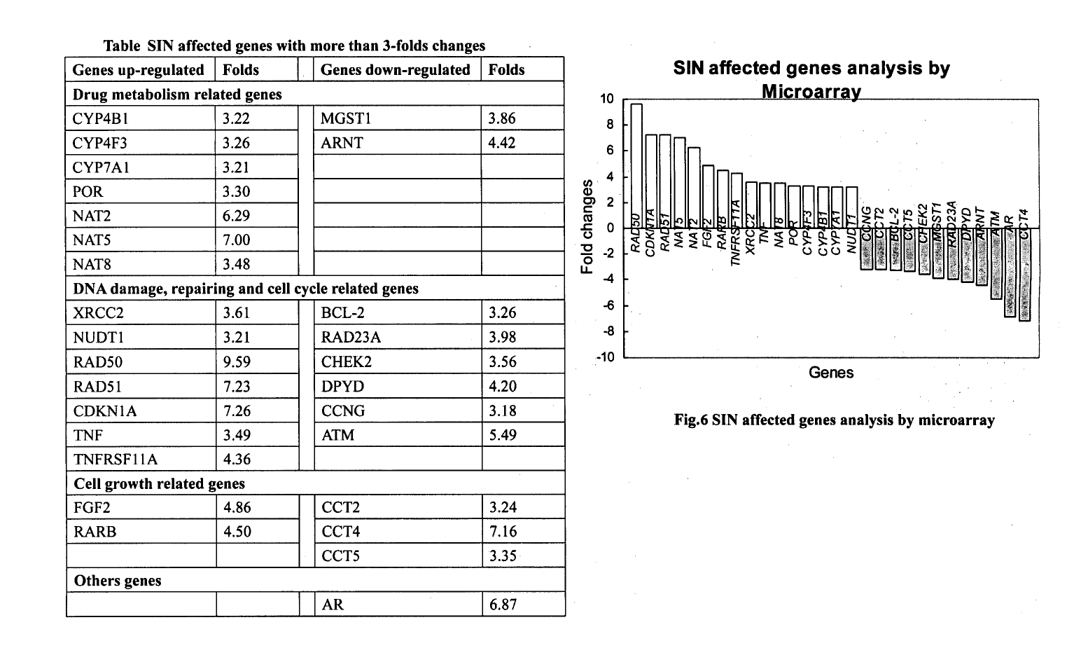

FIG. 5A shows that many genes in drug toxicity and metabolism pathways were differentially expressed in HepG2 cells before and after SIN treatment. FIG. 5B was the image of the control membrane, and FIG. 5C was the image of the SIN treated sample membrane. Table 3.1.4 and FIG. 6 summarized the genes whose expressions were up- or down-regulated more than 3 fold.

### Example 5

### Prevention of HCC Occurrence in Rats by Administering Sinigrin

Sprague Dawley (SD) rats were used as the animal model in the in vivo experiments. Male SD rats (80-90 g body weight) were obtained from Laboratory Animal Services Center of The Chinese University of Hong Kong. The rats were housed in the rodent animal room with a 12 hour light-dark cycle and constant temperature of 25° C. Four rats were housed in each case. Food and water were given ad libitum. All the animals were observed and weighed daily.

Sinigrin Monohydrate was purchased from Fluka. CCl₄ was purchased from Merck. Corn Oil (VeCorn) was purchased from supermarket. DMSO, DEN and Formaldehyde were purchased from Sigma. The reagents used in animal treatment are listed in Table 6 below:

TABLE 6
Reagents Used in Animal Treatment

| SIN solution for oral administration | |
|---|---|
| Sinigrin Monohydrate | 1 g/33.3 ml |
| The solution was made up to 33.3 ml by dH₂O. | |
| CCl₄ solution | |
| CCl₄ | 25 ml/50 ml |
| Corn Oil | 25 ml/50 ml |
| DEN solution (200 mg/ml) | |
| DEN | 2.52 ml/12 ml |
| DMSO | 9.48 ml/12 ml |
| 10% Formaldehyde | |
| Formaldehyde (12.3 M) | 30 ml/108 ml |
| ddH₂O | 78 ml/108 ml |

The experiments were to induce hepatomas in rats. SIN was administered to the rats of treatment group at different stages. Chemical carcinogens were employed in the design of experiment to be able to induce hepatoma within a short time (Ha W. S. et al., 2001). Diethylnitrosamine (DEN) has been frequently used by other scientists to induce hepatoma. DEN administration in combined with CCl₄ method was employed in the experiment to induce hepatoma in rats according to the established protocol (Kovalszky, I. et al., 1992).

DEN is a genotoxic carcinogen that is able to affect all animal species and considered as a probable human carcinogen (Group 2A) (IARC, 1978). DEN catalyzed by P450 and formed an α-hydroxylnitrosamine which was reported to produce mainly liver tumors (IARC, 1978). Carbon Tetrachloride (CCl₄) is a nongenotoxic carcinogen. It can produce liver and mammary neoplasms in rats (IARC, 1999). When it was applied with other carcinogens, the incidence of tumor formation increased (IARC, 1999). This suggests that CCl₄ is a good promoter in the process of hepatoma formation.

Experiments of the promotion stage were to study the effect of SIN at the promotion stage of cancer development. Fifteen rats were randomly divided into 3 groups, a negative control group (5 rats), a positive control group (5 rats) and an SIN-treatment group (5 rats).

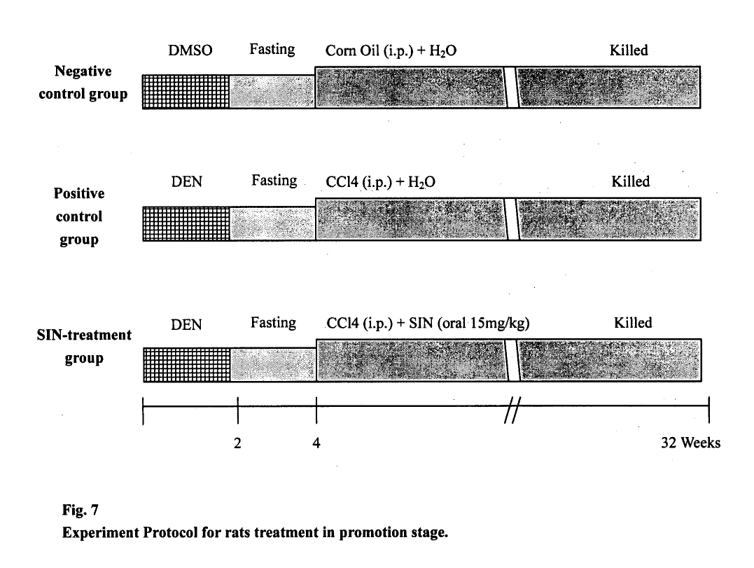

The protocol of the experiment was summarized in FIG. 7.

Table 7 summarized treatment details for the rats.

TABLE 7
Treatment of Rats in Promotion Stage

| | Negative control | Positive control | SIN treatment |
|---|---|---|---|
| Carcinogen | Vehicle | DEN (1 ml/kg) | DEN (200 mg/kg) |
| (i.p.) | (DMSO 1 ml/kg) | | |
| Promoter | Vehicle | CCl₄ (1 ml/kg) | CCl₄ (1 ml/kg) |
| (i.p.) | (Corn Oil 1 ml/kg) | | |
| Treatment | Vehicle (H₂O) | Vehicle (H₂O) | SIN (15 mg/kg) |
| (Oral) | | | |

In the promotion stage experiment, rats in the negative control group received weekly intraperitoneal (i.p.) injection of DMSO for the first 2 weeks and then Corn Oil for the rest of the schedule. Rats in the positive control and SIN-treatment groups both received weekly intraperitoneal (i.p.) injection of the carcinogen DEN for the first 2 weeks and then promoter, Carbon Tetrachloride (CCl₄) for the rest of the schedule. Two weeks of fasting were inserted between the initiator carcinogen treatment and the promoter CCl₄ treatment. During the fasting period of 2 weeks, rats were fasted for 5 days, followed by a 2 day feeding, and another 5 days of fasting. Water was given ad libitum. Negative and positive control groups received oral treatment of water once a day after fasting. Sinigrin with dosage 15 mg/kg was administered orally to the treatment group daily for 28 weeks after fasting.

During the experiment, the body weight of the rat was monitored daily. One week after the last CCl₄ injection, all rats were killed by nitrogen gas asphyxiation and the blood was collected. The rat liver was perfused with ice cold 1×PBS and quickly removed, washed and weighed. The rat livers were photographed for documentation. Five slices of each liver were randomly cut out and the rest of the liver was frozen in liquid nitrogen and stored at −80° C. for further analysis. The liver slices were fixed with 10% formaldehyde solution for 24 hours and stored in 75% ethanol for histological analysis.

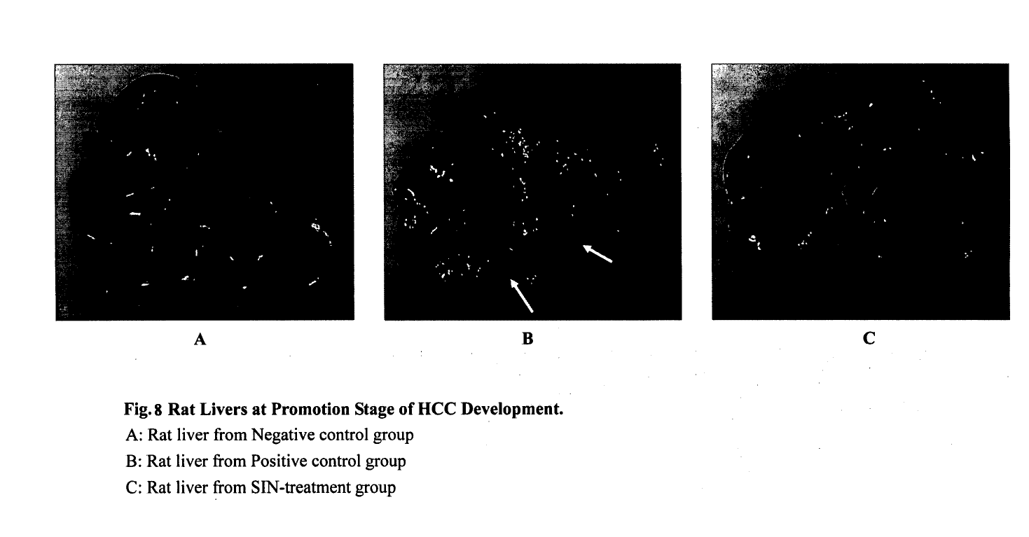

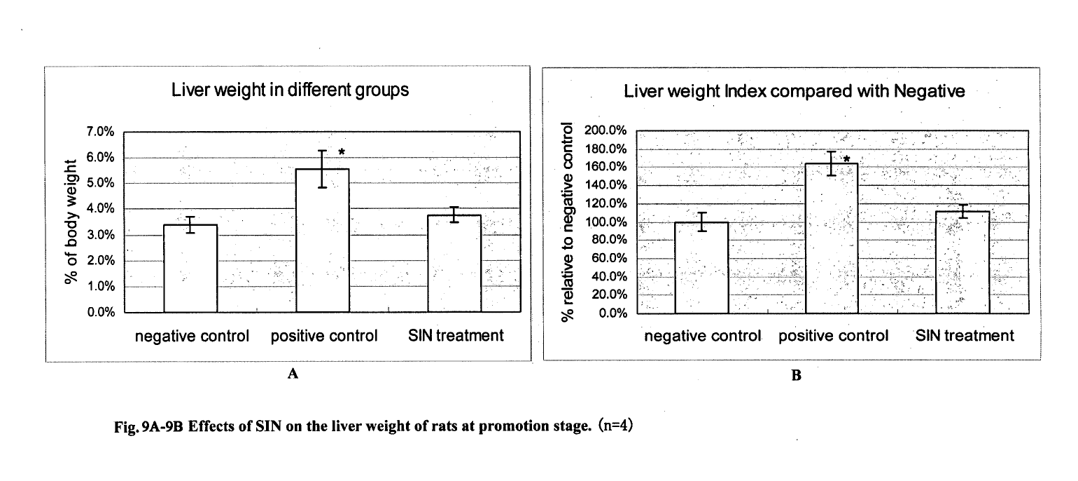

The liver from the negative control group had a healthy appearance (FIG. 8A), but direct observation revealed many tumors on the liver from the positive control group (FIG. 8B). However, there was no visible tumor on the liver from the SIN-treatment group (FIG. 8C). SIN treatment also reversed the liver weight increase which was observed in the positive control group (FIG. 9A, 9B). Compared with the negative group, both the percentage of liver weight and the liver weight index substantially increased in the positive control, while they were only slightly higher in the SIN treatment group (FIG. 9A, 9B). These results indicated that SIN treatment maintained the normal liver morphology.

### Example 6

### AST/ALT Assay at Tumor Progression Stage

ALT (Alanine aminotransferase) and AST (Aspartate aminotransferase) are two enzymes that are specifically located in the liver. These two enzymes normally distribute little in blood serum. When liver damage occurs, the hepatocytes break and these two enzymes are released into the blood. An AST/ALT assay was used to diagnose liver damage. Rat blood was collected in a 13 ml BD PLUS-SST II serum vacutainer and allowed to stand for 20 minutes on ice to coagulate. The vacutainer was then centrifuged at 3,500 rpm for 15 minutes. The upper layer serum was collected. The AST/ALT assay was performed within 2 days to minimize the loss of the enzymes.

AST/ALT (UV-Rate) assay kit was purchased from Stanbio. The vacutainer was purchased from BD Ltd.

The serum AST activity assay was measured by a Stanbio AST (UV-Rate) kit. Fifteen milliliters of ddH₂O were added into one bottle of the reagent in the kit and mixed thoroughly according to the kit protocol. One milliliter of the reagent was incubated at 37° C. for at least 10 minutes for each reaction. One hundred microliters of a sample serum were added to the reagent solution and incubated exactly for 1 minute. Absorbance was measured at OD 340 nm. After incubation for 1 minute, a reading was taken and the reading at this point was counted as time zero. Readings were then taken at 30 second interval for 3 minutes. The change of absorbance per minute was calculated and the AST activity was calculated by the formula

$$( U / L = \frac{Δ A / min}{0.00622} \times \frac{1000 + 100 µl}{100 µl} ) .$$

The ALT assay was measured by a Stanbio AST (UV-Rate) kit. The experimental protocol was the same as for the AST assay.

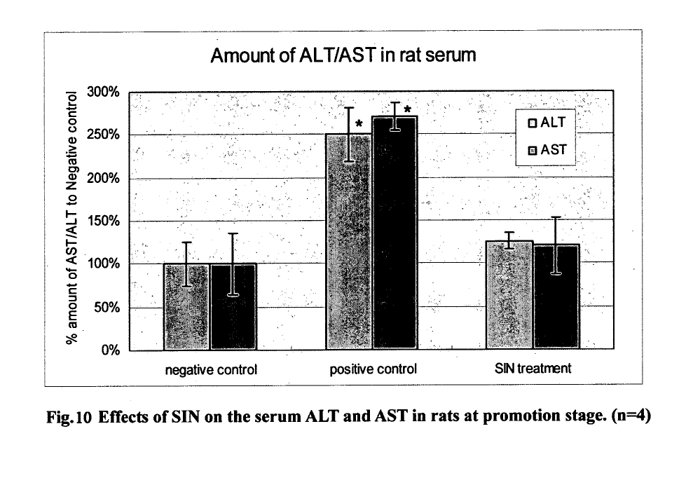

Rat serum ALT and AST levels in the positive control were about 2.5 fold higher than those in the negative control (FIG. 10). SIN treatment lowered ALT and AST amounts to levels similar to those in the negative control (FIG. 10). As serum ALT/AST levels are indicators of liver damage, the results show that SIN treatment can prevent or minimize liver damage.

### Example 7

### SIN Treatment Restored Basic Structure of Hepatocytes at Tumor Promotion Stage

Histological analysis was performed to observe the basic structure of the hepatocytes. Xylene was purchased from Mallinckrodt. Ethanol was purchased from BDH. ABC staining (Anti-rabbit) kit (sc-2018) was purchased from Santa Cruz. Superfrost Microscope slide was purchased from Fisher. 30% H₂O₂, DAB and Formaldehyde were purchased from Sigma. Anti-GST-pi rabbit polyclonal antibody was purchased from MBL. The reagents used are listed in Table 8 below:

TABLE 8
Reagents Used in Histological Analysis

Citric Acid Buffer (pH 6.0)

| | |
|---|---|
| Citric Acid | 0.63 g/300 ml |

The solution was made up to 300 ml by dH₂O and pH was adjusted to 6.0.
Diluted Normal Serum

| | |
|---|---|
| Normal Serum in Kit | 150 μl/10 ml |
| 1X PBS | 9.85 ml/10 ml |

Diluted anti-GST-p antibody (1:10)

| | |
|---|---|
| Anti-GST-p antibody (rabbit polyclonal) | 0.5 ml/5 ml |
| Diluted normal serum | 4.5 ml/5 ml |

Biotinylated goat anti-rabbit IgG (1:200)

| | |
|---|---|
| Biotinylated goat anti-rabbit IgG | 25 μl/5 ml |
| Diluted normal serum | 4.975 ml/5 ml |

Avidin Biotin Enzyme (ABC) mix

| | |
|---|---|
| Reagent A | 100 μl/5 ml |
| Reagent B | 100 μl/5 ml |
| 1X PBS | 4.8 ml/5 ml |

DAB solution

| | |
|---|---|
| DAB | 15 mg/150 ml |
| 30% H₂O₂ | 8 μl/150 ml |

The solution was made up to 150 ml by 1X PBS and filtered before use.

The liver samples were fixed with 10% formaldehyde and put in cassettes for dehydration and fixed with wax. Subsequently, liver samples were embedded in the solidified wax and sliced to 5 μm thickness with a microtome machine. Sliced samples were mounted onto Superfrost microscope slides for further studies.

Slides with liver sections were placed on a slide holder, dewaxed and rehydrated with xylene (3 times, each time 5 minutes), 100% ethanol (3 times, each time 3 minutes), 95% ethanol (3 minutes), 80% ethanol (3 minutes) and distilled water (5 minutes) in order. Slides were then immersed into hematoxylin for about 3 minutes, and the color was allowed to develop for several minutes in water, followed by brief destaining with acid ethanol and water. Slides were immersed into eosin for about 30 seconds to stain the cytoplasm red, followed by washing in tap water for several minutes. Slide-checking was necessary to assure the best staining effect. Slides were then dehydrated with 95% ethanol (3 minutes), 100% ethanol (3 times, each time 5 minutes), and xylene (3 times, each time 5 minutes), in order. Finally, the slides were mounted with coverslips and allowed to dry. Axiophot-2 Universal microscope (Zeiss) connected to a computer. The images were captured by Spot 32 image capture software (Diagnostic Instruments).

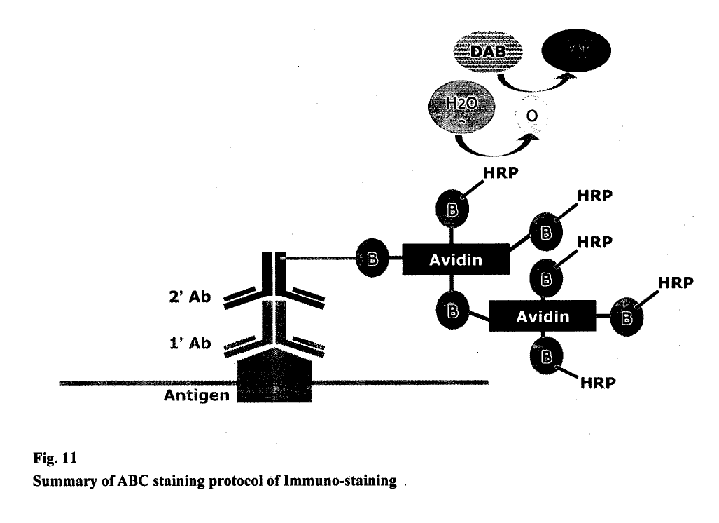

Slides with liver sections were placed on a slide holder, dewaxed and rehydrated with xylene and different concentrations of ethanol as mentioned previously. Slides were incubated with 1% H₂O₂ solution for 5 minutes at room temperature to block endogenous peroxidase activity followed by washing in tap water for 5 minutes. A hot citric acid buffer (pH 6.0) was freshly prepared. Slides were heated in the hot citric acid buffer for 5 minutes for antigen retrieval. Slides were washed with three changes of PBS for 5 minutes each. The slides were removed from PBS and placed in a moist chamber. The immunostaining was performed with a Santa Cruz ABC staining kit. FIG. 11 summarizes the overall design of the ABC kit. Two hundred microliters of Diluted Normal Serum solution were added to the sections on each slide and allowed to incubate for 1 hour to block nonspecific binding. After draining the normal serum, 200 μl diluted Anti-GST-pi antibody (rabbit polyclonal, 1:10) was then applied and incubated overnight at 4° C. After washing with 1×PBS for 3 times, 5 minutes each, 200 μl diluted biotinylated secondary antibody (1:200) was applied and incubated for 30 minutes at room temperature. After three changes of PBS for 5 minutes each, 200 μl avidin & biotinylated horseradish peroxidase macromolecular complex (ABC) was added to incubate for 30 minutes. Slides were washed with PBS and visualized by DAB solution for approximately 3 minutes. After washing with tap water for several minutes, slides were counterstained in hematoxylin for 3 minutes and washed with tap water. Slides were dehydrated as mentioned previously. Finally, the slides were mounted with coverslips and allowed to dry.

The images were checked and captured by an Axiophot-2 Universal microscope (Zeiss) connected with computer for documentation. The image was recorded by Spot 32 image capture software (Diagnostic Instruments). The image with 2.5 fold magnification was used to calculate the GST-p positive area/whole area ratio by the analysis software Image J.

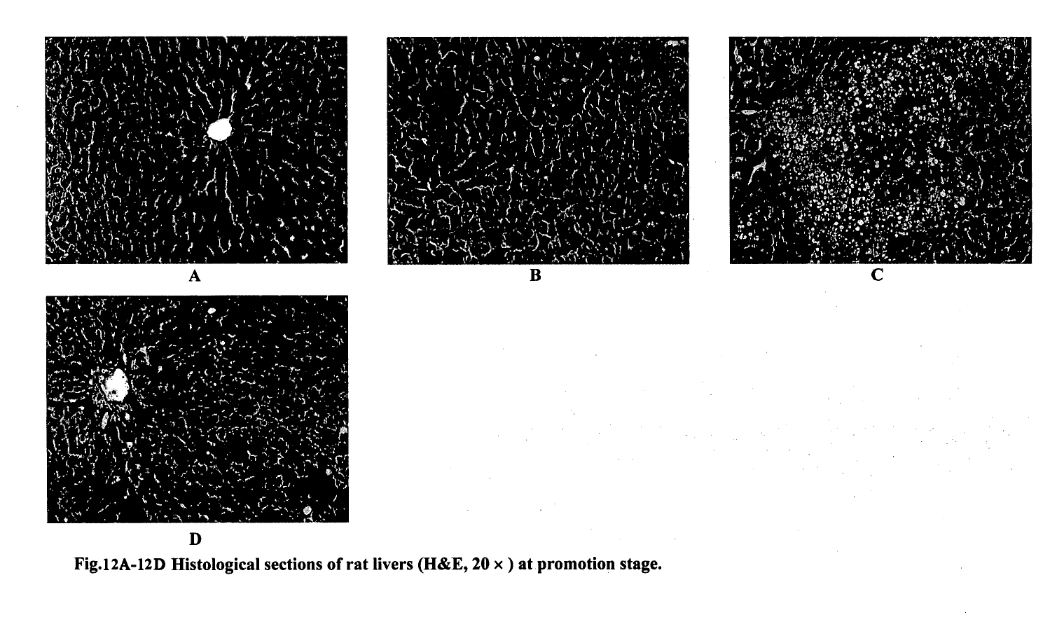

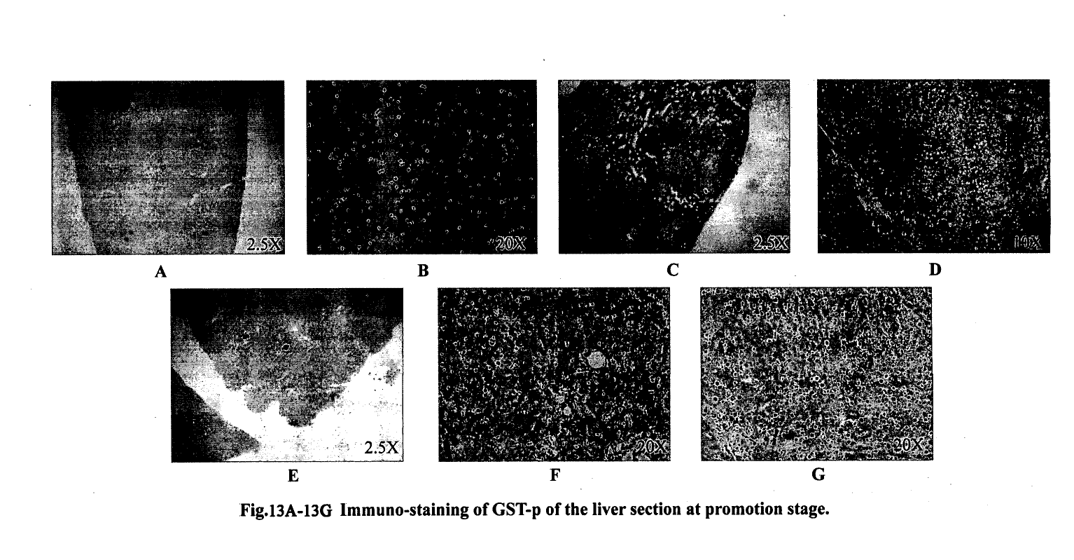

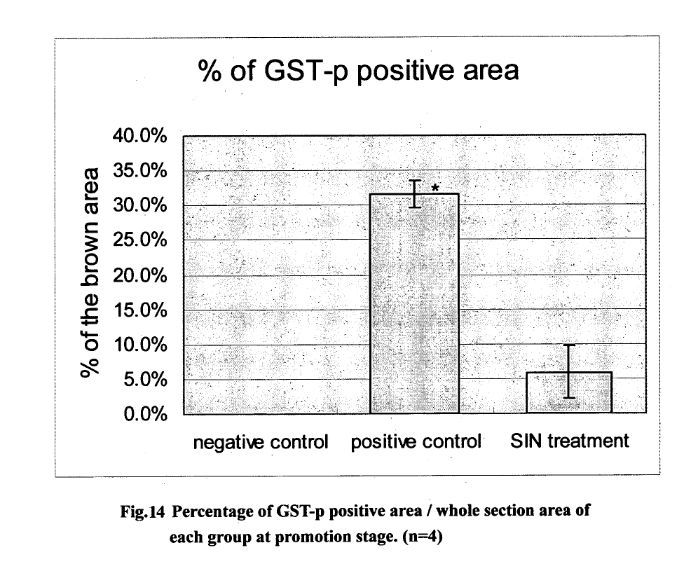

FIG. 12A showed the normal basic structure of hepatocytes of the liver of the negative control: both the cells and the nuclei were well defined and the cells were bright red. However, hepatocytes were severely damaged in livers from the positive control group. The cells were dark red and the central vein was lost (FIG. 12B). Moreover, cytoplasmic vacuolization within the hepatocytes could be observed (FIG. 12C). In the livers from the SIN-treatment group, the basic structure of hepatocytes and the central vein were restored (FIG. 12D). No GST-p expression was found in livers from the negative control (FIG. 13A, B). However, immunostaining of GST-p revealed extended clustered GST-p positive areas in the positive control (FIG. 13C, D). Only limited GST-p positive area was found in livers from the SIN-treatment group (FIG. 13E, F), implying that SIN treatment restored the basic structure of hepatocytes. FIG. 14 compared the percentage of the GST-p positive area of each group. SIN treatment reduced the GST-p positive area from 35% in the positive control to only 5% in the testing group.

### Example 8

### Gene Expression Assay at Promotion Stage

Expression patterns, at both mRNA and protein levels, of selected genes related to apoptosis and tumors were examined to investigate the mechanism by which sinigrin prevent tumor occurrence.

Oligo dT primer, 10 mM dNTP mix, Taq polymerase, SuperScript II Reverse Transcriptase and β-actin primer mix were purchased from Invitrogen. 5× first strand buffer, and 10×PCR buffer was purchased from GibcoBRL. MgCl₂ was purchased from Sigma. Target gene, primer mixes were purchased from Tech Dragon Ltd. The reagents used are listed in Table 9 below:

TABLE 9
Reagents Used in the Gene Expression Assay

| 25 mM MgCl₂ | |
|---|---|
| MgCl₂ | 0.119 g/50 ml |
| The solution was made up to 50 ml by ddH₂O. | |
| 0.1 M DTT | |
| 0.5 M Dithiothreitol (DTT) | 10 ml/50 ml |
| ddH₂O | 40 ml/50 ml |

RNA was extracted from 300 μg of rat liver and was measured as described in foregoing examples Three micrograms of total RNA, 1 μl of an oligo dT primer and certain volume of RNase-Free H₂O were mixed into a total volume of 12 μl. The mixture was incubated at 70° C. for 10 minutes and immediately chilled on ice. Four microliters of 5× First Stand Buffer, 2 μl of 0.1M DTT, 1 μl of 10 mM dNTP mix, and 1 μl of SuperScript II were added to each tube and mixed. The tubes were incubated at 42° C. for 50 minutes followed by 70° C. for 15 minutes before chilling on ice. The cDNA was then ready for PCR amplification.

The PCR reactions were performed in a final volume of 20 μl in a Gene Amp® PCR system. The PCR reaction mix contained 1 μl of cDNA synthesized previously, 2 μl 10×PCR Buffer, 0.4 μl 10 mM dNTP mix, 1.2 μl 25 mM MgCl₂, 1 μl 10 mM primer mix, 0.2 μl recombinant Taq polymerase, and 14.2 μl autoclaved ddH₂O. The detail of primer mix of each gene was summarized in Table 10 below:

TABLE 10
Primer sequences for PCR

| | | |
|---|---|---|
| Gene | Orientation | 5′ Primer sequence 3′ |

| | | |
|---|---|---|
| β-actin | Forward | ACA CCT CAA ACC ACT CCC AG (SEQ ID NO: |
| | | 1) |
| | Reverse | AAC TCC TAA GGG GAG GAT GG (SEQ ID |
| | | NO: 2) |
| p53 | Forward | GTGG ATCC TGAA GACT GGAT AACT GTC |
| | | (SEQ ID NO: 3) |
| | Reverse | AGTC GACA GGAT GCAG AGGC TG (SEQ ID |
| | | NO: 4) |
| Mdm2 | Forward | GTCT CTGG ACTC GGAA GATT AC (SEQ ID |
| | | NO: 5) |
| | Reverse | AAAC AATG CTGC TGGA AGTC G (SEQ ID |
| | | NO: 6) |

For synthesis of β-actin gene as the internal control and Mdm2 gene, the PCR mixture was incubated at 94° C. for 5 minutes followed by 30 cycles of amplification. Each cycle consisted of 45 seconds of denaturation at 94° C., 45 seconds of annealing at 55° C. and 30 seconds of extension at 72° C. After all cycles were completed, a final extension step at 72° C. for 10 minutes was performed. The PCR products were ready for gel electrophoresis.

For synthesis of p53, the number of cycle was enhanced to 35, the annealing temperature was enhanced to 58° C., and the extension time was extended to 1.5 minutes, respectively.

PCR products were visualized on a DNA gel as described previously.

The intensity of the PCR products was analyzed by the software Image J. The band intensity of the β-actin gene was measured as an internal control. The ratio of the band intensities of the target gene to the β-actin gene was used to compare the mRNA level of each gene between different treatment groups.

HEPES, MgCl₂, Glycerol, DTT, Acrylamide, N,N′-Methylenebisacrylamide, β-mercaptoethanol and EDTA were purchased from Sigma. Tris-base, Bromophenol Blue, Triton-X-100, NaCl, PMSF, Glycine and SDS powder were purchased from USB. ECL detection kit was purchased from AMERSHAM. The protease inhibitor tablet was purchased from ROCHE. Methanol was purchased from BDH. Tween-20 was purchased from Pharmacia Biotech. Immobilon-P PVDF membrane was purchased from Millipore. Primary antibodies including anti-Mdm2 mouse monoclonal SMP14, anti-p53 mouse monoclonal Pab 246, anti-Bcl-2 mouse monoclonal c-2, anti-Bax mouse monoclonal 5B7, anti-p21 mouse monoclonal, and anti-PCNA mouse monoclonal antibodies were purchased from Santa Cruz, Calif. Anti-wild type p53 mouse monoclonal Ab6 was purchased from Oncogene Science. Secondary antibodies including goat anti-mouse IgG and goat anti-rabbit IgG antibodies were purchased from Santa Cruz, Calif. Super RX X-Ray Film was purchased from FujiFilm Ltd. Reagents used in Western blot analysis are listed below in Table 11 below:

TABLE 11
Reagents Used in Western Blot Analysis

Solution A (pH 7.9)

| | |
|---|---|
| Triton X-100 | 0.3 g/50 ml |
| HEPES | 0.11915 g/50 ml |
| 0.5M EDTA (pH 8.0) | 0.1 ml/50 ml |
| NaCl | 0.4385 g/50 ml |
| 0.5 mM PMSF | 125 μl/50 ml |
| Protease inhibitor | 1 tablet/50 ml |

The solution was made up to 50 ml by ddH₂O and pH was adjusted to
7.9.
Solution B (pH 7.9)

| | |
|---|---|
| Glycerol | 12.5 ml/50 ml |
| HEPES | 238.8 mg/50 ml |
| 0.5M EDTA (pH 8.0) | 20 μl/50 ml |
| NaCl | 1.2272 g/50 ml |
| MgCl₂ | 5.712 mg/50 ml |
| 0.5 mM Dithiothreitol (DTT) | 50 μl/50 ml |
| 0.5 mM PMSF | 250 μl/50 ml |
| Protease inhibitor | 1 tablet/50 ml |

The solution was made up to 50 ml by ddH₂O and the pH was adjusted
to 7.9.
Solution C (pH 7.9)

| | |
|---|---|
| Triton X-100 | 0.3 ml/50 ml |
| Glycerol | 12.5 ml/50 ml |
| 0.2M HEPES | 5 ml/50 ml |
| 0.5M EDTA (pH 8.0) | 20 μl/50 ml |
| NaCl | 0.1785 g/50 ml |
| MgCl₂ | 5.7 mg/50 ml |
| 0.5 mM Dithiothreitol (DTT) | 62.5 μl/50 ml |
| 0.5 mM PMSF | 250 μl/50 ml |
| Protease inhibitor | 1 tablet/50 ml |

The solution was made up to 50 ml by ddH₂O and the pH was adjusted
to 7.9.
30% Acrylamide mix

| | |
|---|---|
| Acrylamide | 29 g/100 ml |
| N,N′-Methylenebisacrylamide | 1 g/100 ml |

The solution was stored at 4° C. in the dark.
2xSDS sample loading dye

| | |
|---|---|
| Tris-HCl (1 M, pH 6.8) | 2.5 ml/25 ml |
| 10% SDS | 10 ml/25 ml |
| Bromophenol blue | 0.00625 g/25 ml |
| Glycerol (99%) | 5 ml/25 ml |
| β-mercaptoethanol (14.4 M) | 2.5 ml/25 ml |

The solution was made up to 25 ml with ddH₂O.
1xSDS Running Buffer

| | |
|---|---|
| Tris-base | 3.02 g/L |
| Glycine | 18.8 g/L |
| 10% SDS | 10 ml/L |

10% Resolving gel (for one gel)

| | |
|---|---|
| ddH₂O | 3.6 ml |
| 30% acrylamide mix | 2.96 ml |
| 1M Tris (pH 8.8) | 2.25 ml |
| 10% SDS | 75 μl |
| 10% APS | 75 μl |
| TEMED | 3.5 μl |

3% Stacking gel

| | |
|---|---|
| ddH₂O | 2.55 ml |
| 30% acrylamide mix | 0.68 ml |
| 1.5M Tris (pH 6.8) | 0.48 ml |
| 10% SDS | 37.5 μl |
| 10% APS | 37.5 μl |
| TEMED | 3.75 μl |

Transfer buffer

| | |
|---|---|
| Tris-base | 5.82 g/1 L |
| Glycine | 2.93 g/1 L |
| 20% Methanol | 200 ml/1 L |
| 10% SDS | 3.75 ml/1 L |

The solution was made up to 1 L with ddH₂O.
TBST buffer (pH 7.5)

| | |
|---|---|
| 1 M Tris | 10 ml/1 L |
| 5 M NaCl | 30 ml/1 L |
| 100% Tween-20 | 1 ml/1 L |

The solution was made up to 1 L with ddH₂O and the pH was adjusted
to 7.5.

Three hundred micrograms of each liver sample were homogenized in the Solution C at 1 g/ml using a glass homogenizer on ice. The homogenate was transferred to a microcentrifuge tube and centrifuged at 13,000 rpm for 20 minutes. The supernatant was collected and equal volume of 2× sample loading dye was added to each sample. The samples were stored at −20° C. The protein was used for immunodetection of the expression level of Bax, Bcl-2, PCNA and p21.

Three hundreds micrograms of each liver sample were homogenized in Solution A at 1 g/ml using the glass homogenizer on ice. The homogenate was transferred into a microcentrifuge tube and centrifuged at 3,000 rpm for 5 minutes to spin down the unbroken tissue. The supernatant was collected and incubated on ice for 5 minutes and then centrifuged at 5,000 rpm for 5 minutes. The supernatant was discarded and the pellet was lysed by 100 μl Solution B. The lysing process lasted for 20 minutes on ice. After incubation, the solution was centrifuged at 12,000 rpm for 15 seconds to spin down the cell debris. The supernatant was collected and equal volume of 2× sample loading dye was added to each sample. The samples were ready for immunodetection of the expression level of p53 and mdm2.

The Mini-PROTEAN II electrophoresis cell (Bio-Rad) was used for sodium dodecyl sulfate poly acrylamide gel electrophoreses (SDS-PAGE). The gel mold was assembled according to the protocol. About 3 ml water was poured into the set mold to test for leakage. After discarding the water, resolving gel solution was prepared according to the established protocol. A 10% acrylamide gel was used for the separation. The resolving gel was poured into the mold at a proper volume (about ⅔ full). After the gel solution was poured into the mold, a thin layer of distilled water was poured on the gel solution to ensure the surface of the gel was smooth. The gel was allowed to polymerize for 30 minutes. Distilled water at the top of the gel was then removed. The stacking gel solution was poured on top of the resolving gel The comb for sample slots was inserted into the stacking gel immediately. The stacking gel was allowed to polymerize for more than 30 minutes. The whole gel setup was placed in a gel tank. Fresh 1×SDS running buffer was then added into the tank, over the gel setup. The comb was then removed. Protein samples with loading buffer were added into the slots in the stacking gel. The gel was run under 150V for 1 hour until the dye reached the end of the gel.

The gel was removed from the glass plates and immersed in the transfer buffer. PVDF membrane (Immobilon-P, Millipore) was used for Western blot. The membrane was first wet in methanol for 1 minute. The membrane was submersed in H₂O and transfer buffer for several minutes, respectively. Six pieces of filter paper cut to the same size of the gel were soaked in the transfer buffer. Three pieces of filter paper were placed on the bed of the transfer tank. Air bubbles were removed. After that, the membrane was placed on the top. Then the gel and another three pieces of wetted filter paper were placed on the membrane. The transfer process was at 10 V for 1 hour (mA limit: 4 mA/cm² gel). The excess transfer buffer was removed by washing in TBST buffer for several minutes. The membranes were ready for antibody blocking.

Each of the transferred membrane blots was incubated in 4 ml TBST with 5% non-fat milk powder and primary antibody overnight at 4° C. After incubation, the membrane was washed in TBST buffer for 10 minutes for 3 times. Each transferred membrane blot was then incubated in 4 ml TBST and 5% non-fat milk powder with secondary antibody for 1 hour at room temperature. After incubation, the membrane was washed in TBST buffer for 10 minutes for 3 times. The membrane was ready for ECL reaction.

The ECL reagent was stored at 4° C. 0.5 ml of Solution 1 and 0.5 ml of Solution 2 of the ECL kit (Amersham) were freshly mixed before use for each blot. The blot was contacted with the mixed solution for 5 min. Two blots can successively be developed with the same solution. The membrane was wrapped in Saran wrap and exposed to X-ray film immediately for different periods of time. The exposure time depended on the strength of the antibody.

The films that captured the signal from the ECL reaction were scanned into the computer and analyzed by the software Image J to read the density of darkness.

DNA fragmentation has suggested that SIN treatment can induce apoptosis of HepG2 cells (FIG. 4), which can be a mechanism by which SIN suppresses cancer. Therefore, it would be intriguing to examine expression patterns of apoptosis-related genes, such as p53 and Bcl-2, as well as genes related to oncogenesis, including Mdm2, p21, PCNA, and Bax. Gene expression was examined at mRNA level or protein level or both. As shown in FIGS. 13-17, SIN treatment reversed the expression changes in the positive control group in most cases, which may be due to the mechanisms of sinigrin's tumor suppressing function.

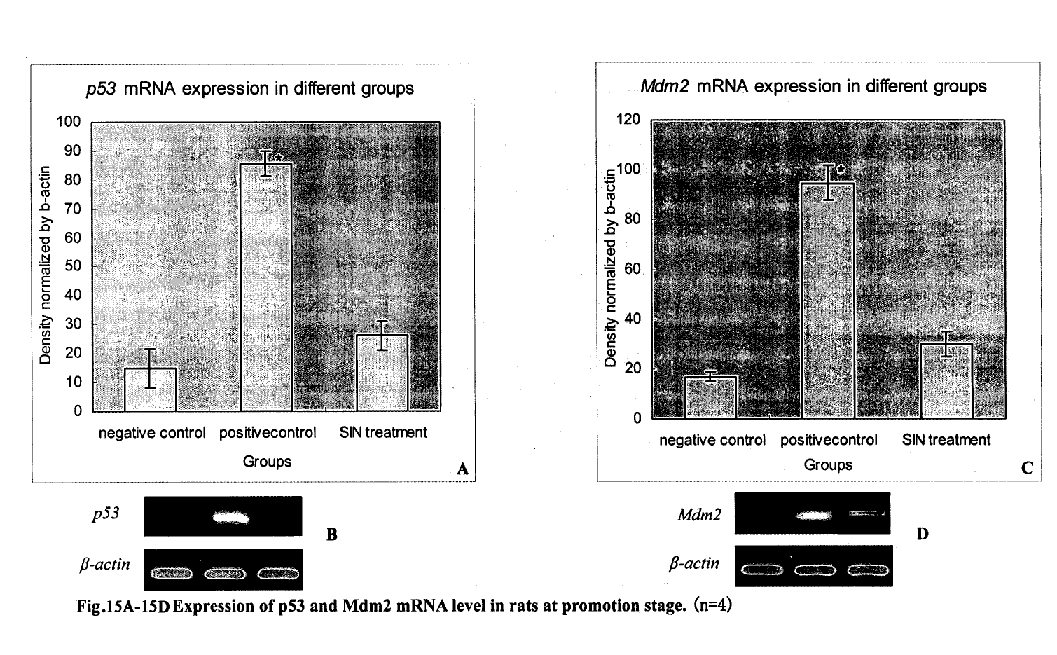

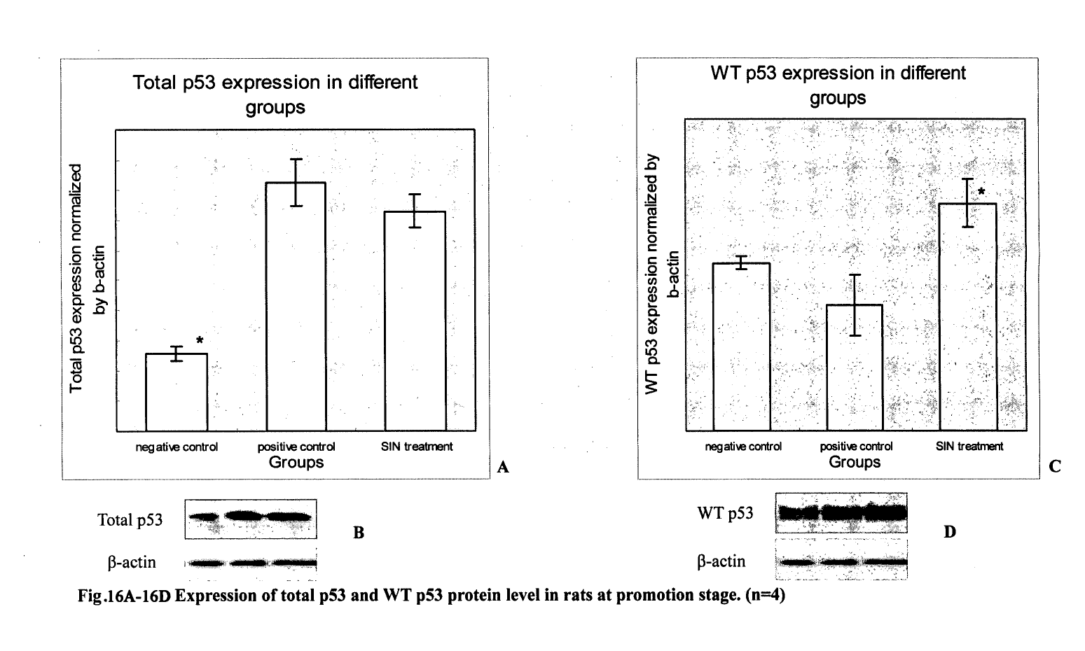

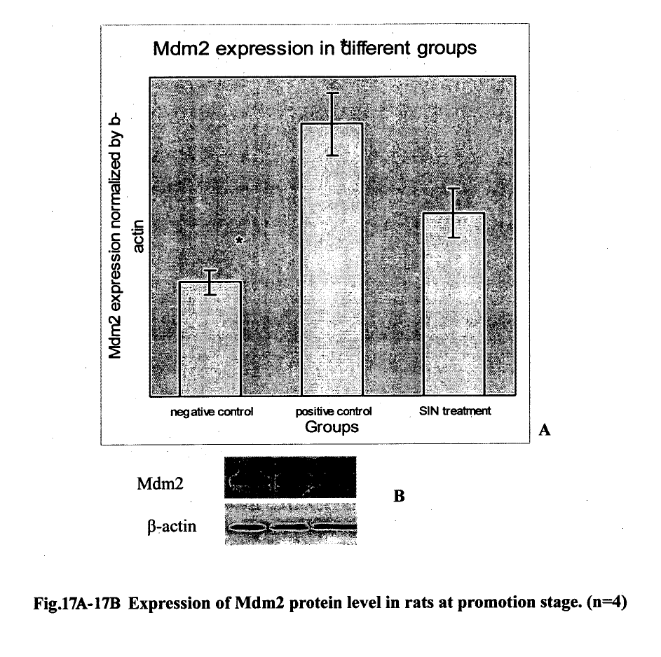

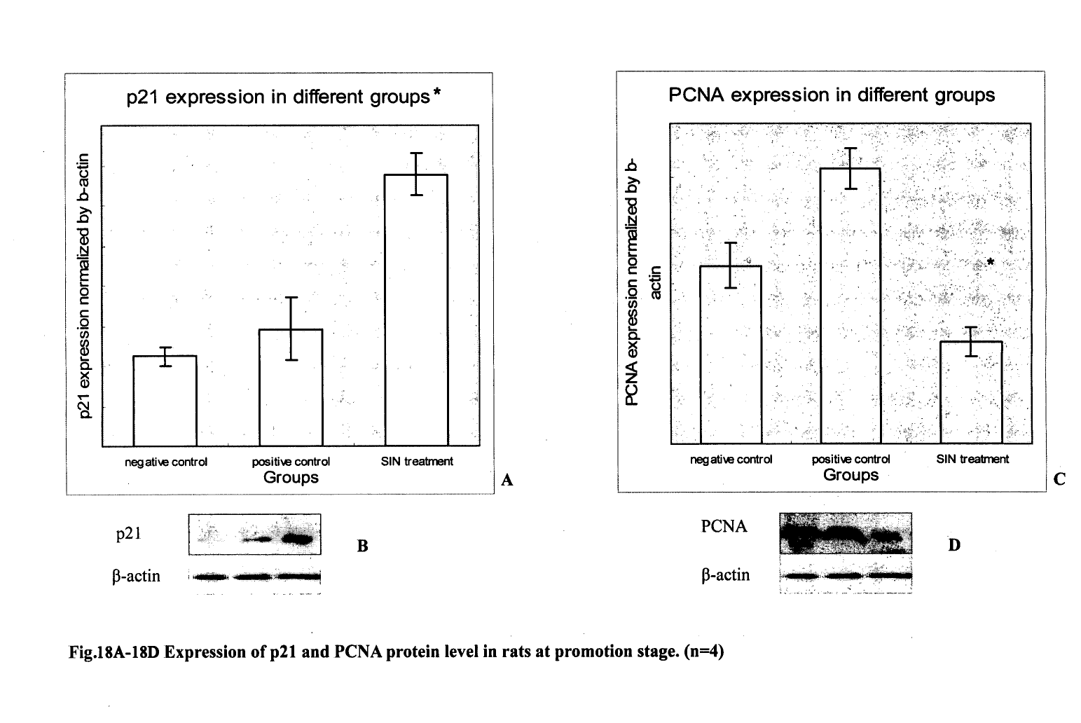

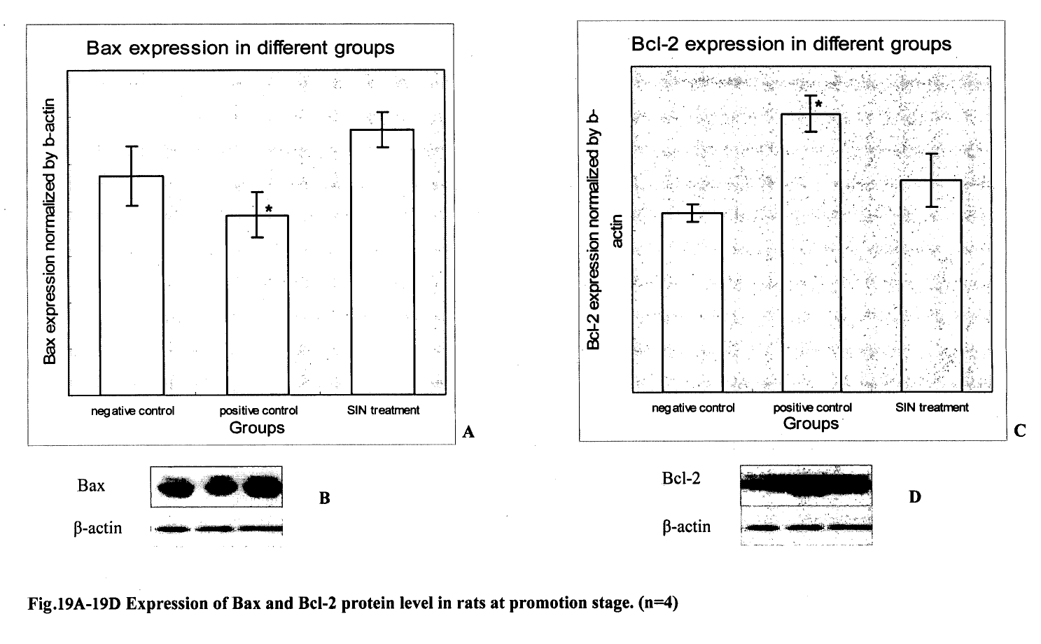

In particular, mRNA expressions of both p53 and Mdm2 were substantially increased in the positive control, while their expressions in the SIN treatment group were decreased back to levels near the negative control (FIG. 15). Over-expression of p53 is often observed in tumors, but some of them are mutated p. 53. FIG. 16A showed that although the level of total p53 protein significantly increased in the positive control and SIN treatment group (FIG. 16A, B), the level of the wild type p53 protein decreased in the positive control and SIN treatment restored its expression to a level even higher than the negative control (FIG. 16C, D). Mdm2 protein expression was significantly increased in the positive control, and SIN treatment partially reversed it (FIG. 17). SIN treatment also reversed the increased expressions of PCNA protein and Bcl-2 protein and the decreased expression of Bax protein in the positive group (FIG. 18C, D; FIG. 19). However, surprisingly, the expression of p21 protein in the SIN treatment group was much higher than that of the negative control and the positive control (FIG. 18A, B). This result may imply the complexity of the mechanism by which SIN suppresses tumor growth.

### Example 9

### Cure of HCC in Rats by Administering Sinigrin

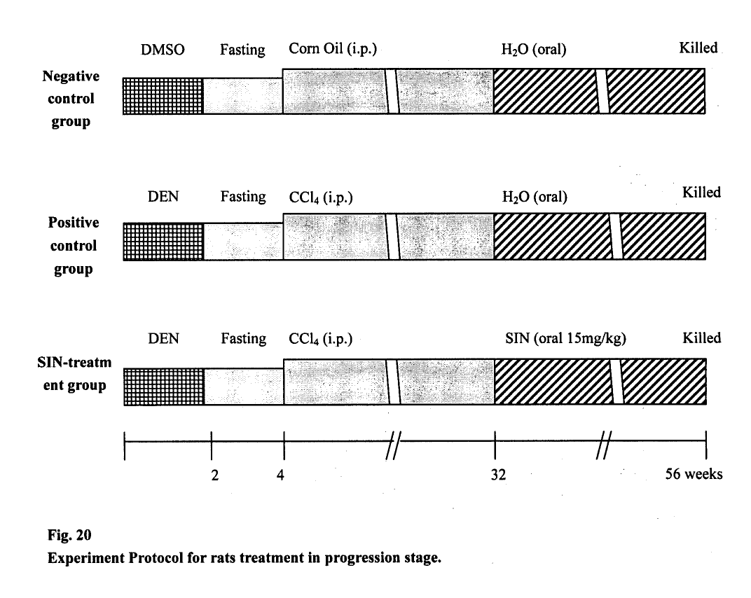

The effect of SIN on progression stage of cancer development was studied. FIG. 20 summarized the protocol applied to rats. The amounts of rats and grouping method were all the same as in the promotion stage experiment.

Rats in the negative control group received weekly intraperitoneal (i.p.) injection of DMSO for the first 2 weeks and then Corn Oil for 28 weeks. Rats in the positive control and SIN-treatment groups both received weekly intraperitoneal (i.p.) injection of DEN for the first 2 weeks and then CCl₄ for 28 weeks. Two weeks of fasting were inserted between DEN treatment and CCl₄ treatment. Starting from 33^{rd} week, negative and positive control groups received oral administration of water once a day. Sinigrin with dosage 15 mg/kg was administered orally to the SIN-treatment group daily for 24 weeks. After treatment, the rats were killed. Blood and liver were collected and processed as described in the foregoing examples.

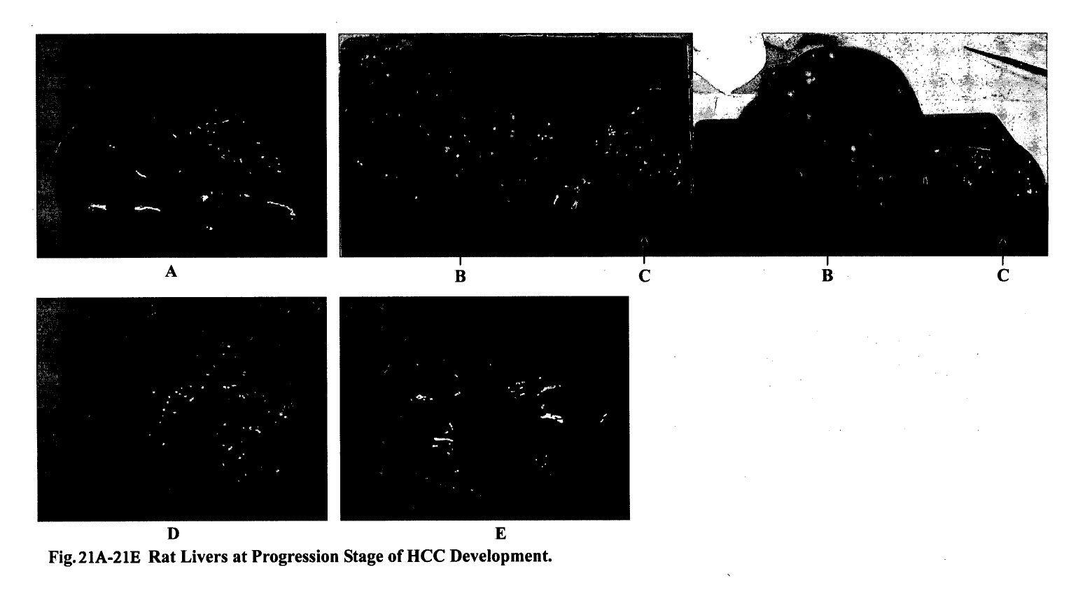

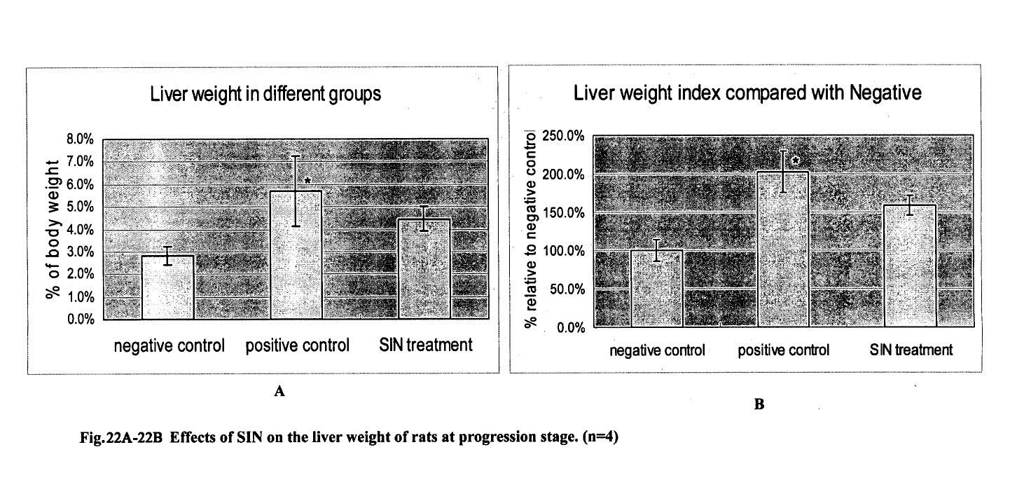

The liver from the negative control group had a healthy appearance (FIG. 21A), and direct observation revealed a large tumor on the liver from the positive control group resulting in loss of its normal liver shape (FIG. 21B, C). However, there were few visible tumors on the livers from the SIN-treatment group (FIG. 21D). SIN treatment also reversed the liver weight increase which was observed in the positive control group (FIG. 22). Compared to the negative group, both the percentage of liver weight and the liver weight index substantially increased in the positive control, while they were only slightly higher in the SIN treatment group (FIG. 22). These results indicate that SIN treatment restores the normal morphology of liver.

### Example 10

### AST/ALT Assay at Progression Stage

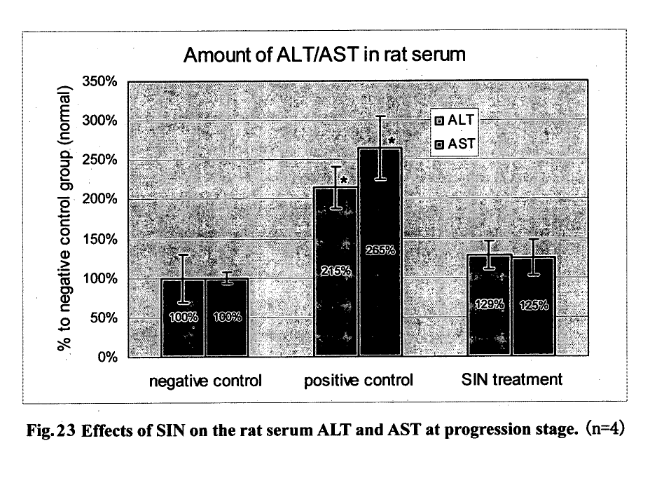

AST/ALT assay was performed as described in the foregoing examples. Rat serum ALT and AST levels in the positive control were 215% and 265% of those in the negative control, respectively (FIG. 23). SIN treatment lowered ALT and AST amounts to levels similar to those in the negative control (FIG. 23). As serum ALT/AST levels are indicators of liver damage, the results show that SIN treatment can, at least partially, reverse liver damage.

### Example 11

### SIN Treatment Restored Basic Structure of Hepatocytes at Progression Stage

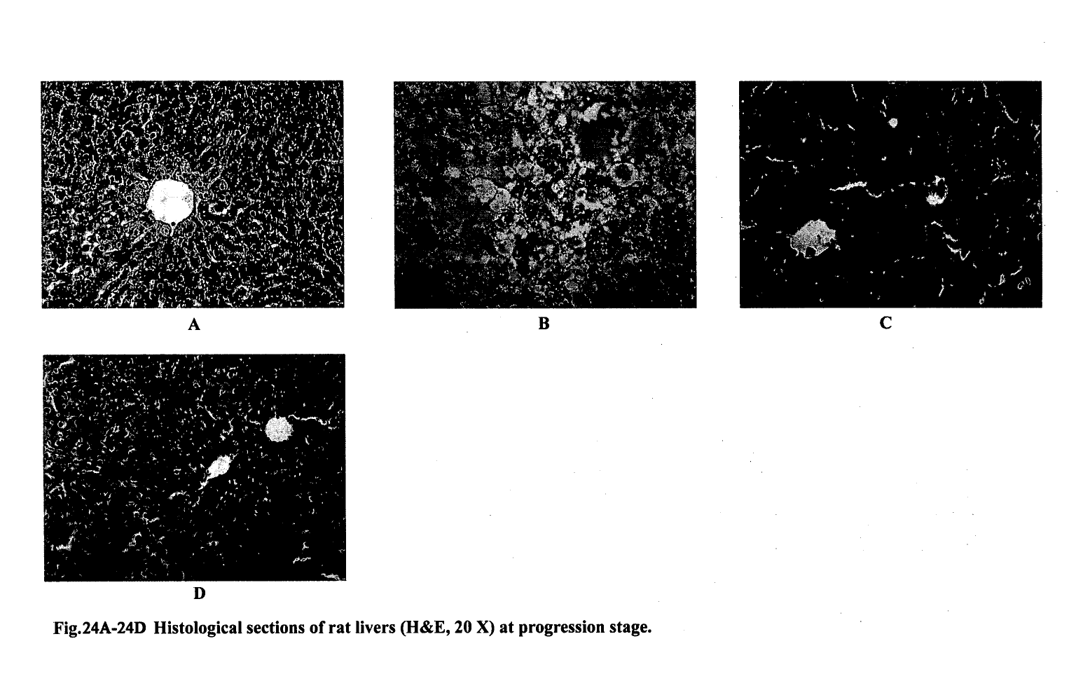

The experiments were performed as described in the foregoing examples. FIG. 24A shows the normal basic structure of hepatocytes of the liver of the negative control: both the cells and the nuclei were well defined and the cells were bright red. However, the hepatocyte was severely damaged in livers from the positive control group as shown by the presented clusters of fatty droplets (FIG. 24B). Moreover, cell death and the increased blood vessels and blood cytoplasmic vacuolization within the hepatocytes could be observed (FIG. 24C). In the liver from the SIN-treatment group, the basic structure was restored without abnormal appearance (FIG. 24D).

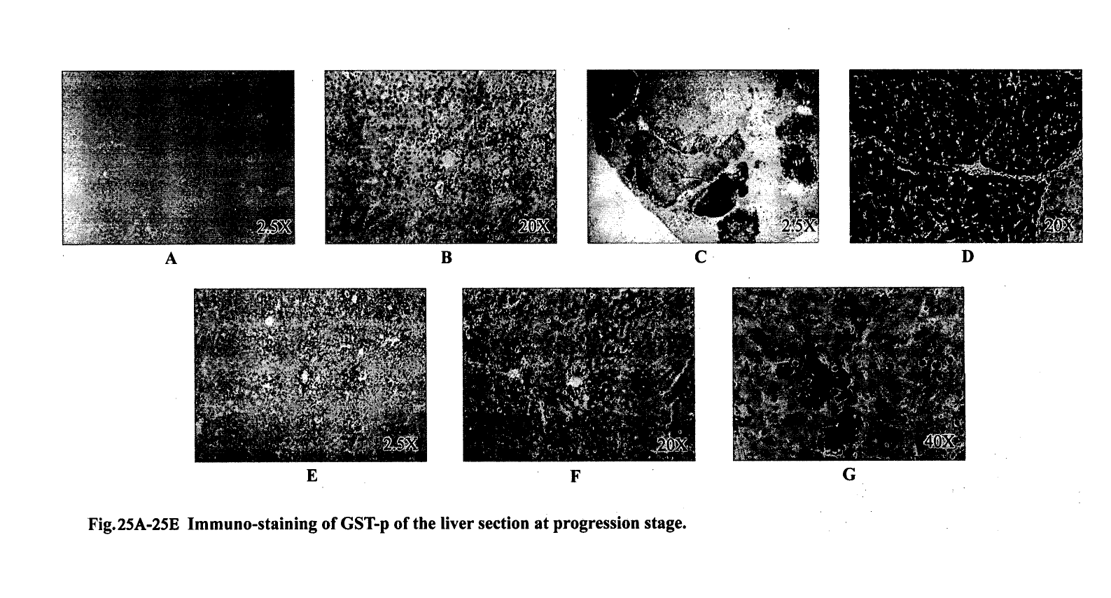

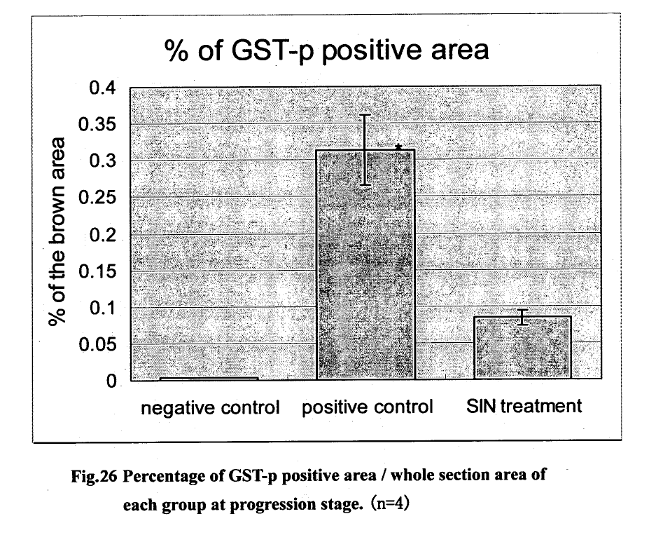

No GST-p expression was found in the liver from the negative control (FIG. 25A, 25B). However, immunostaining of GST-p found clustered GST-p positive areas across the section in the positive control (FIG. 25C). Necrotic cells also could be observed (FIG. 25D). Only limited GST-p positive area was found in the liver from the SIN-treatment group (FIG. 25E, 25F, 25G), demonstrating that SIN treatment can restore the basic structure of hepatocytes. FIG. 26 compared the percentage of the GST-p positive area of each group. SIN treatment reduced the GST-p positive area from about 30% in positive control to less than 10% in the SIN treatment group.

### Example 12

### Gene Expression Assay at Progression Stage

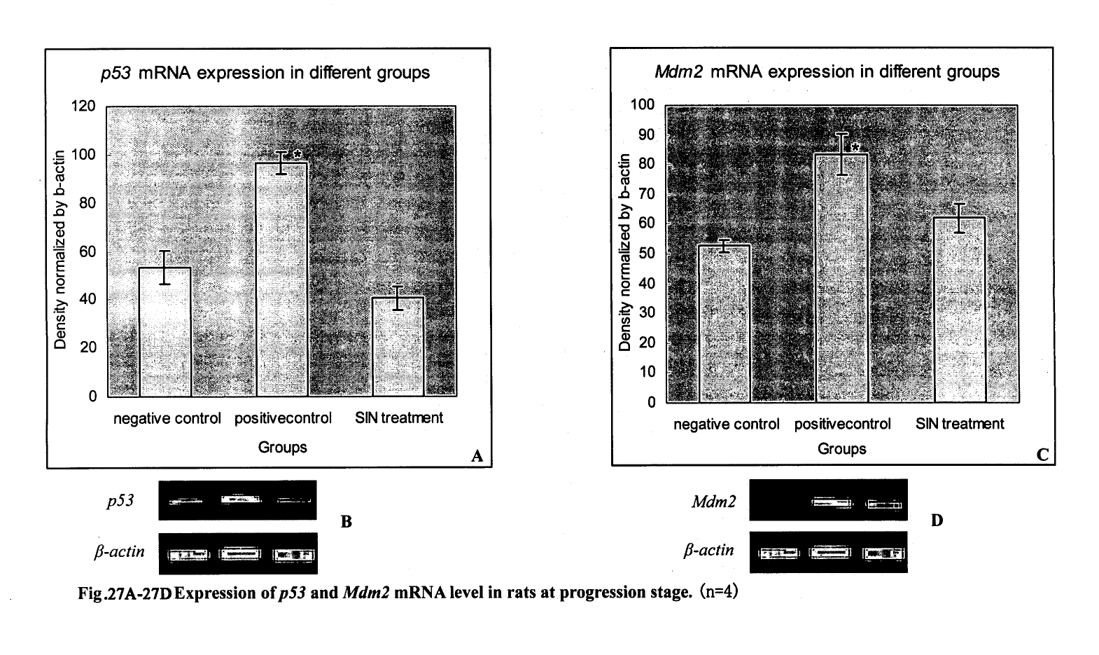

The gene expression assays were performed as described in the foregoing examples. Expression patterns of selected apoptosis- and oncogenesis-related genes, including p53, Mdm2, p21, PCNA, Bax and Bcl-2, were examined by semi-quantitative PCR and Western Blotting at mRNA level or protein level or both. As shown in FIGS. 27-31, SIN treatment reversed the expression changes of these genes in the positive control group in most cases, which could be suggestive to the mechanisms of sinigrin's tumor suppressing function.

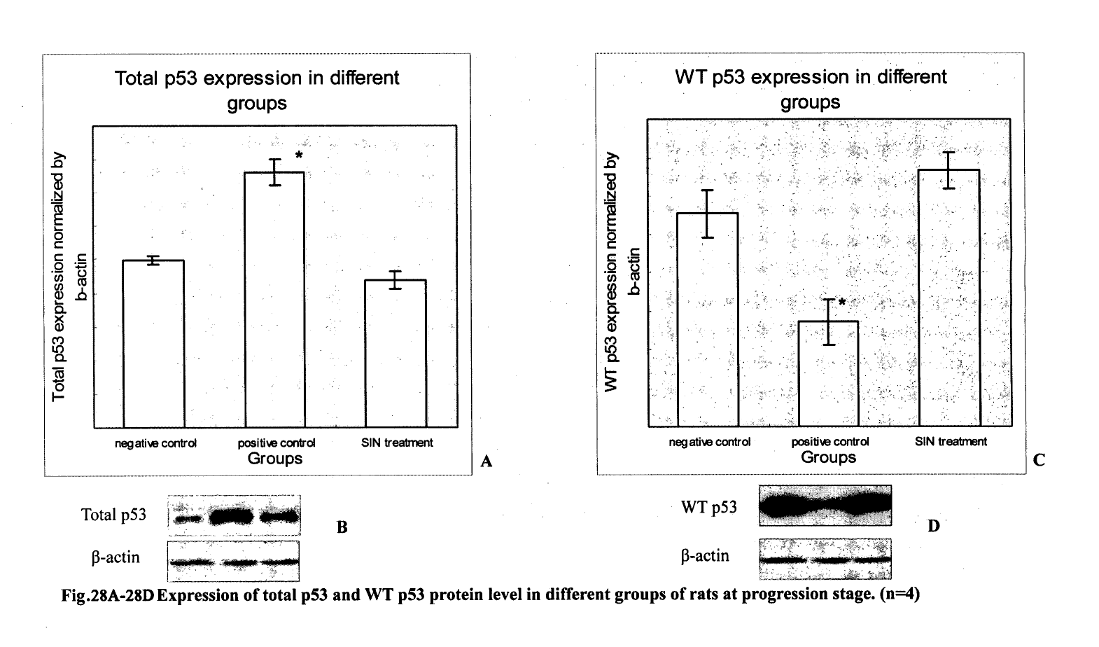

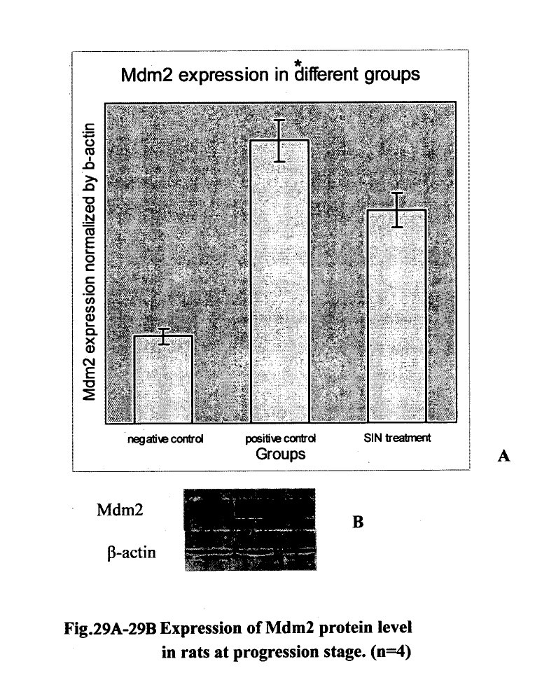

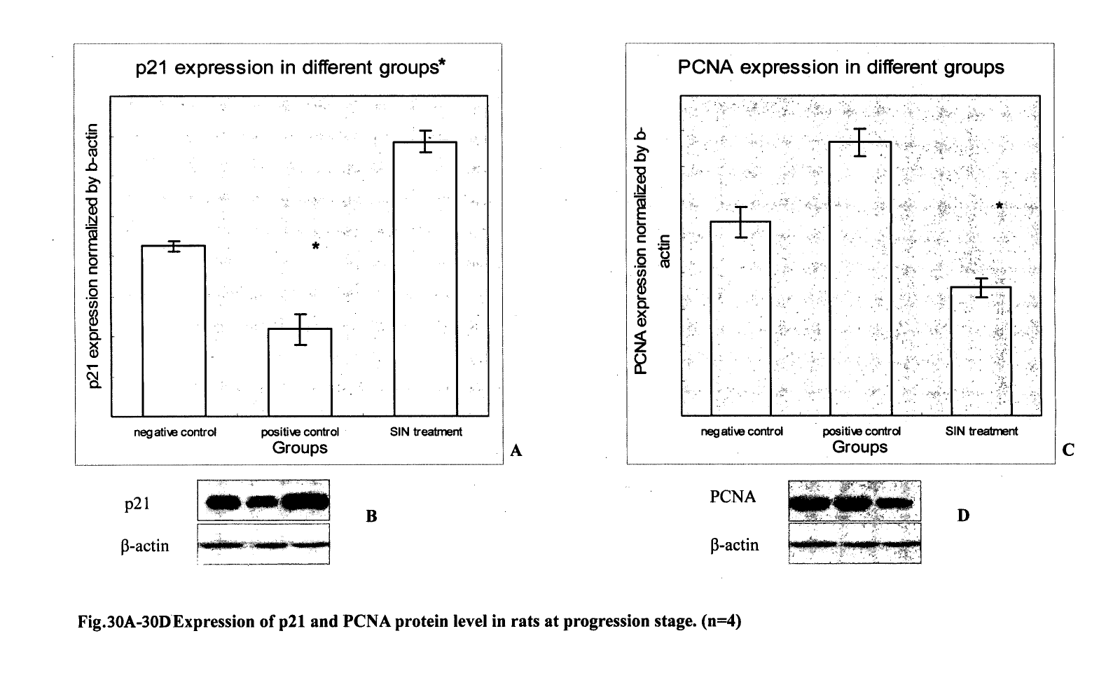

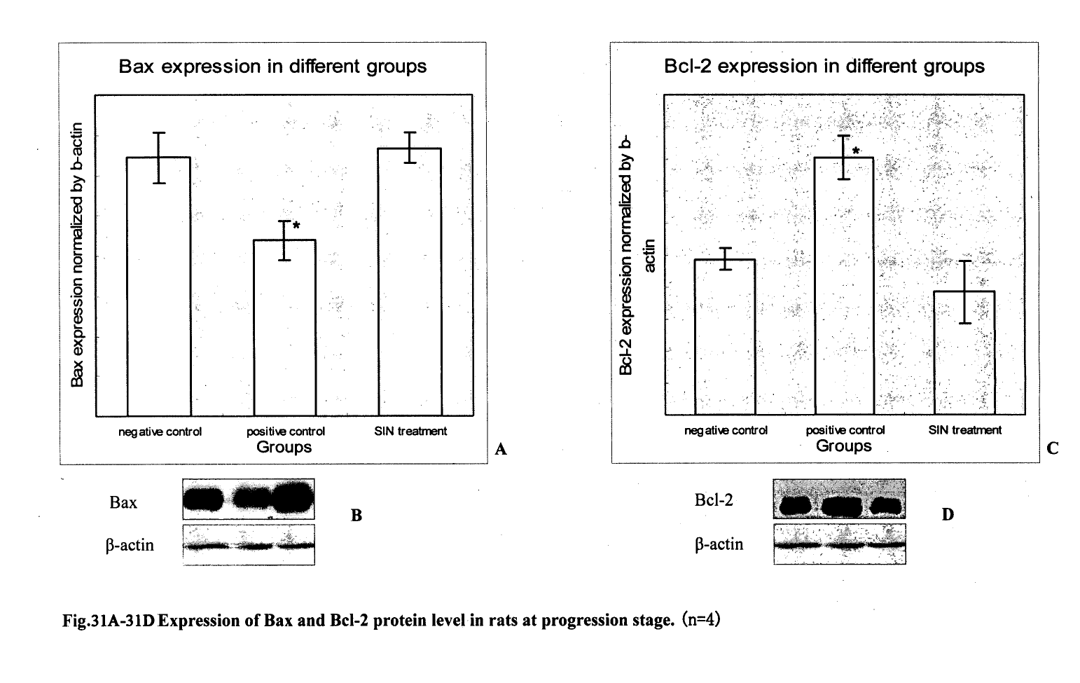

In particular, mRNA expressions of both p53 and Mdm2 were significantly increased in the positive control, while their expressions in the SIN treatment group were reduced back to levels near or even lower than the negative control (FIG. 27). Over-expression of p53 is often observed in tumors, but some of them are mutated p53. FIG. 28A showed that although the level of total p53 protein significantly increased in the positive control and SIN treatment group (FIG. 28A, B), the level of the wild type p53 protein decreased in the positive control and SIN treatment restored its expression to a level even higher than that of the negative control (FIG. 28C, D). Mdm2 protein expression was significantly increased in the positive control, and SIN treatment partially reversed it partially (FIG. 29). SIN treatment also reversed the increased expressions of PCNA protein and Bcl-2 protein and the decreased expression of Bax protein in the positive group (FIG. 30C, D; FIG. 31). Differ from the situation of promotion stage, p21 protein expression decreased in the positive control as expected, and SIN treatment restored its expression to a level substantially higher than the negative control (FIG. 30A, B).

From the above description of the invention, those skilled in the art will perceive improvements, changes and modifications. Such improvements, changes and modifications within the skill of the art are intended to be covered by the appended claims.

### REFERENCES

1. Alshatwi A A, Han C T, Schoene N W, &Lei KY. (2006) Nuclear Accumulations of p53 and Mdm2 Are Accompanied by Reductions in c-Abl and p300 in Zinc-Depleted Human Hepatoblastoma Cells. Exp Biol Med (Maywood). 231(5):611-618.
2. Babich, H. & Borenfreund, E. (1990) Applications of the neutral red cytotoxicity assay to in vitro toxicology (Review). Alternatives to Laboratory Animals, 18, 129-144.
3. Gutieerrez-Ruiz M C, Bucio L, Souza V, Gomez J J, Campos C & Carabez A. (1994) Expression of some hepatocyte-like functional properties of WRL-68 cells in culture. In Vitro Cell Dev Biol Anim. 30A (6):366-71.
4. Ha W S, Kim C K, Song S H, Kang C B. (2001) Study on mechanism of multistep hepatotumorigenesis in rat: development of hepatotumorigenesis. J Vet Sci. 2(1):53-8.
5. Hugh J M Brady. (2004) Apoptosis Method and Protocols. Volume 282, Page 13. Humana Press.
6. Kovalszky I, Kovalszky I, Szeberenyi S, Zalatnai A, Vincze I, Lapis K, Jeney A. (1992) Modification of DENA-induced hepatocarcinogenesis by CCl4 cirrhosis. Comparison of the marker enzyme patterns. Carcinogenesis. 13(5):773-8.
7. Remington's Pharmaceutical Sciences, 18th ed., Mack Publishing Co., Easton, Pa. (1990)
8. Johnson I., et al. Colon cancer proliferation desulfosinigrin in Wasabi (Wassabia japonica). Nutrition and Cancer. 2004; 48(2):207
9. Zheng, Q, et al. Further investigation of the modifying effect of various chemopreventive agents on apoptosis and cell proliferation in human colon cancer cells. Journal of Cancer Research and Clinical Oncology, 2002; 128:539-546

1. A pharmaceutical composition for treating liver cancer consisting of sinigrin or a pharmaceutically acceptable derivate thereof or a mixture of both, and a pharmaceutically acceptable carrier.

2. The composition of claim 1, wherein the pharmaceutical composition comprises a therapeutically effective amount of sinigrin or a pharmaceutically acceptable derivate thereof or a mixture of both which is in the range of from about 1 mg/kg to about 100 mg/kg body weight daily.

3. The composition of claim 2, wherein the therapeutically effective amount is 10 mg/kg body weight daily.

4. The composition of claim 1, wherein the pharmaceutically acceptable carrier is water.

5. The composition of claim 1, wherein the pharmaceutical composition is formulated in a unit dosage form selected from the group consisting of tablets, dragees, troches, capsules, solution solutions, or suspensions, depots and sustained release formulations.

6. The composition of claim 5, wherein the composition comprises a dosage unit of sinigrin or a pharmaceutically acceptable derivate thereof or a mixture of both in the range of about 0.1 mg to about 100 mg.

7. The composition of claim 5, wherein the unit dosage is in the range of about 1 mg to about 15 mg.

8. The composition of claim 5, wherein the unit dosage is 1 mg, 2.5 mg, 5 mg or 10 mg.

9. The composition of claim 1, wherein the cancer is a primary cancer or a secondary cancer.

10. (canceled)

11. The composition of claim 1, wherein the pharmaceutical composition reduces viability of tumor cells or induces apoptosis of tumor cells.

12. (canceled)

13. The composition of claim 1, wherein the pharmaceutical composition changes expression of an apoptosis-related gene selected from the group consisting of p53, mdm2, p21, pcna, bax and bcl-2.

14. The composition of claim 1, wherein the pharmaceutical composition changes mRNA expression of a gene in drug toxicity and metabolism pathways, wherein the gene is at least one gene selected from the group consisting of cyp4b1, cyp4f3, cyp7a1, por, nat2, nat5, nat8, mgst1, arnt, xrcc2, nudt1, rad50, rad51, cdkn1a, tnf, tnfrsf11a, bcl-2, rad23a, chek2, dpyd, ccng, atm, fgf2, rarb, cct2, cct4, cct5, and ar.

15. A method for treating liver cancer in a subject, comprising administering to the subject a therapeutically effective amount of a pharmaceutical composition of claim 1 or sinigrin or a pharmaceutically acceptable derivate thereof or a mixture of both.

16. The method of claim 15, wherein the cancer is a primary cancer or a secondary cancer.

17. (canceled)

18. The method of claim 15, wherein the pharmaceutical composition is administered to the subject intravenously, intraperitoneally, subcutaneously, intramuscularly, orally, topically or intratumorally.

19. (canceled)

20. The method of claim 15, wherein the sinigrin or a pharmaceutically acceptable derivate thereof or a mixture of both is administrated in a therapeutically effective amount in the range of from about 1 mg/kg to about 100 mg/kg body weight daily.

21. The method of claim 20, wherein the sinigrin or a pharmaceutically acceptable derivate thereof or a mixture of both is administrated in a therapeutically effective amount of 10 mg/kg body weight daily.

22. (canceled)

23. The method of claim 15, wherein the subject is a human.

24. A method of inhibiting growth of hepatoma cells or inducing apoptosis of hepatoma cells comprising contacting the hepatoma cells with a therapeutically effective amount of the pharmaceutical composition of claim 1 or sinigrin or a pharmaceutically acceptable derivate thereof or a mixture of both.

25. (canceled)

26. The method of claim 24, wherein the hepatoma cells are human hepatoma cells.

27-43. (canceled)
